(19) United States
(12) Patent Application Publication
(10) Pub. No.: US 2007/0213311 A1
(43) Pub. Date: Sep. 13, 2007

(54) Modulators of 11-beta hydroxyl steroid dehydrogenase type 1, pharmaceutical compositions thereof, and methods of using the same

(76) Inventors: Yun-Long Li, Chadds Ford, PA (US); Wenqing Yao, Kennett Square, PA (US)

Correspondence Address: FISH & RICHARDSON PC, P.O. BOX 1022, MINNEAPOLIS, MN, 55440-1022, US

(21) Appl. No.: 11/712,708

(22) Filed: Mar. 1, 2007

Related U.S. Application Data
(60) Provisional application No. 60/778,312, filed on Mar. 2, 2006.
(60) Provisional application No. 60/809,035, filed on May 26, 2006.

(51) Int. Cl. A61K 31/5375; A61K 31/53; A61K 31/52; A61K 31/519; A61K 31/517; A61K 31/47

(52) U.S. Cl. 514/183; 514/241; 514/255.06; 514/263.1; 514/266.1; 514/265.1; 514/249; 514/310; 514/311; 514/256; 514/355; 514/365; 514/374; 514/367; 514/471; 514/237.5; 514/255.01; 514/210.17; 514/317; 514/423; 514/613; 544/162; 544/182; 544/277; 544/280; 544/406; 544/335; 544/392; 544/283; 544/353; 546/315; 546/226; 548/950; 548/954; 548/530; 564/188; 564/189; 564/190; 546/146; 546/176

(57) ABSTRACT

The present invention relates to inhibitors of 11-β hydroxyl steroid dehydrogenase type 1 and pharmaceutical compositions thereof. The compounds of the invention can be useful in the treatment of various diseases associated with expression or activity of 11-β hydroxyl steroid dehydrogenase type 1.

### CROSS REFERENCE TO RELATED APPLICATIONS

This application claims the benefit of U.S. Ser. Nos. 60/778,312, filed Mar. 2, 2006, and 60/809,035, filed May 26, 2006, the disclosures of which are incorporated herein by reference in their entireties.

### FIELD OF THE INVENTION

The present invention relates to modulators of 11-β hydroxyl steroid dehydrogenase type 1 (11βHSD1), compositions thereof, and methods of using the same.

### BACKGROUND OF THE INVENTION

Glucocorticoids are steroid hormones that have the ability to modulate a plethora of biological processes including development, neurobiology, inflammation, blood pressure, and metabolism. In humans, the primary endogenously produced glucocorticoid is cortisol. Two members of the nuclear hormone receptor superfamily, glucocorticoid receptor (GR) and mineralcorticoid receptor (MR), are the key mediators of cortisol function in vivo. These receptors possess the ability to directly modulate transcription via DNA-binding zinc finger domains and transcriptional activation domains. This functionality, however, is dependent on the receptor having first bound to ligand (cortisol); as such, these receptors are often referred to as ‘ligand-dependent transcription factors.’

Cortisol is synthesized in the zona fasciculate of the adrenal cortex under the control of a short-term neuroendocrine feedback circuit called the hypothalamic-pituitary-adrenal (HPA) axis. Adrenal production of cortisol proceeds under the control of adrenocorticotrophic hormone (ACTH), a factor produced and secreted by the anterior pituitary. Production of ACTH in the anterior pituitary is itself highly regulated, being driven by corticotropin releasing hormone (CRH) produced by the paraventricular nucleus of the hypothalamus. The HPA axis functions to maintain circulating cortisol concentrations within restricted limits, with forward drive at the diurnal maximum or during periods of stress being rapidly attenuated by a negative feedback loop resulting from the ability of cortisol to suppress ACTH production in the anterior pituitary and CRH production in the hypothalamus.

The importance of the HPA axis in controlling glucocorticoid excursions is evident from the fact that disruption of this homeostasis by either excess or deficient secretion or action results in Cushing's syndrome or Addison's disease, respectively (Miller and Chrousos (2001) Endocrinology and Metabolism, eds. Felig and Frohman (McGraw-Hill, New York), 4^{th} Ed.: 387-524). Interestingly, the phenotype of Cushing's syndrome patients closely resembles that of Reaven's metabolic syndrome (also known as Syndrome X or insulin resistance syndrome) including visceral obesity, glucose intolerance, insulin resistance, hypertension, and hyperlipidemia (Reaven (1993) Ann. Rev. Med. 44: 121-131). Paradoxically, however, circulating glucocorticoid levels are typically normal in metabolic syndrome patients.

For decades, the major determinants of glucocorticoid action were believed to be limited to three primary factors: 1) circulating levels of glucocorticoid (driven primarily by the HPA axis), 2) protein binding of glucocorticoids in circulation (upward of 95%), and 3) intracellular receptor density inside target tissues. Recently, a fourth determinant of glucocorticoid function has been identified: tissue-specific pre-receptor metabolism. The enzymes 11-beta hydroxysteroid dehydrogenase type 1 (11βHSD1) and 11-beta hydroxysteroid dehydrogenase type 2 (11βHSD2) catalyze the interconversion of active cortisol (corticosterone in rodents) and inactive cortisone (11-dehydrocorticosterone in rodents). 11βHSD1 has been shown to be an NADPH-dependent reductase, catalyzing the activation of cortisol from inert cortisone (Low et al. (1994) J. Mol. Endocrin. 13: 167-174); conversely, 11βHSD2 is an NAD-dependent dehydrogenase, catalyzing the inactivation of cortisol to cortisone (Albiston et al. (1994) Mol. Cell. Endocrin. 105: R11-R17). The activity of these enzymes has profound consequences on glucocorticoid biology as evident by the fact that mutations in either gene cause human pathology. For example, 11βHSD2 is expressed in aldosterone-sensitive tissues such as the distal nephron, salivary gland, and colonic mucosa where its cortisol dehydrogenase activity serves to protect the intrinsically non-selective mineralcorticoid receptor from illicit occupation by cortisol (Edwards et al. (1988) Lancet 2: 986-989). Individuals with mutations in 11βHSD2 are deficient in this cortisol-inactivation activity and, as a result, present with a syndrome of apparent mineralcorticoid excess (also referred to as ‘SAME’) characterized by hypertension, hypokalemia, and sodium retention (Wilson et al. (1998) Proc. Natl. Acad. Sci. 95: 10200-10205). Likewise, mutations in 11βHSD1 and a co-localized NADPH-generating enzyme, hexose 6-phosphate dehydrogenase (H6PD), can result in cortisone reductase deficiency (also known as CRD; Draper et al. (2003) Nat. Genet. 34: 434-439). CRD patients excrete virtually all glucocorticoids as cortisone metabolites (tetrahydrocortisone) with low or absent cortisol metabolites (tetrahydrocortisols). When challenged with oral cortisone, CRD patients exhibit abnormally low plasma cortisol concentrations. These individuals present with ACTH-mediated androgen excess (hirsutism, menstrual irregularity, hyperandrogenism), a phenotype resembling polycystic ovary syndrome (PCOS).

Given the ability of 11βHSD1 to regenerate cortisol from inert circulating cortisone, considerable attention has been given to its role in the amplification of glucocorticoid function. 11βHSD1 is expressed in many key GR-rich tissues, including tissues of considerable metabolic importance such as liver, adipose, and skeletal muscle, and, as such, has been postulated to aid in the tissue-specific potentiation of glucocorticoid-mediated antagonism of insulin function. Considering a) the phenotypic similarity between glucocorticoid excess (Cushing's syndrome) and the metabolic syndrome with normal circulating glucocorticoids in the later, as well as b) the ability of 11βHSD1 to generate active cortisol from inactive cortisone in a tissue-specific manner, it has been suggested that central obesity and the associated metabolic complications in syndrome X result from increased activity of 11βHSD1 within adipose tissue, resulting in ‘Cushing's disease of the omentum’ (Bujalska et al. (1997) Lancet 349: 1210-1213). Indeed, 11βHSD1 has been shown to be upregulated in adipose tissue of obese rodents and humans (Livingstone et al. (2000) Endocrinology 131: 560-563; Rask et al. (2001) J. Clin. Endocrinol. Metab. 86: 1418-1421; Lindsay et al. (2003) J. Clin. Endocrinol. Metab. 88: 2738-2744; Wake et al. (2003) J. Clin. Endocrinol. Metab. 88: 3983-3988).

Additional support for this notion has come from studies in mouse transgenic models. Adipose-specific overexpression of 11βHSD1 under the control of the aP2 promoter in mouse produces a phenotype remarkably reminiscent of human metabolic syndrome (Masuzaki et al. (2001) Science 294: 2166-2170; Masuzaki et al. (2003) J. Clinical Invest. 112: 83-90). Importantly, this phenotype occurs without an increase in total circulating corticosterone, but rather is driven by a local production of corticosterone within the adipose depots. The increased activity of 11βHSD1 in these mice (2-3 fold) is very similar to that observed in human obesity (Rask et al. (2001) J. Clin. Endocrinol. Metab. 86: 1418-1421). This suggests that local 11βHSD1-mediated conversion of inert glucocorticoid to active glucocorticoid can have profound influences whole body insulin sensitivity.

Based on this data, it would be predicted that the loss of 11βHSD1 would lead to an increase in insulin sensitivity and glucose tolerance due to a tissue-specific deficiency in active glucocorticoid levels. This is, in fact, the case as shown in studies with 11βHSD1-deficient mice produced by homologous recombination (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). These mice are completely devoid of 11-keto reductase activity, confirming that 11βHSD1 encodes the only activity capable of generating active corticosterone from inert 11-dehydrocorticosterone. 11βHSD1-deficient mice are resistant to diet- and stress-induced hyperglycemia, exhibit attenuated induction of hepatic gluconeogenic enzymes (PEPCK, G6P), show increased insulin sensitivity within adipose, and have an improved lipid profile (decreased triglycerides and increased cardio-protective HDL). Additionally, these animals show resistance to high fat diet-induced obesity. Further, adipose-tissue overexpression of the 11-beta dehydrogenase enzyme, 11βHSD2, which inactivates intracellular corticosterone to 11-dehydrocorticosterone, similarly attenuates weight gain on high fat diet, improves glucose tolerance, and heightens insulin sensitivity. Taken together, these transgenic mouse studies confirm a role for local reactivation of glucocorticoids in controlling hepatic and peripheral insulin sensitivity, and suggest that inhibition of 11βHSD1 activity may prove beneficial in treating a number of glucocorticoid-related disorders, including obesity, insulin resistance, hyperglycemia, and hyperlipidemia.

Data in support of this hypothesis has been published. Recently, it was reported that 11βHSD1 plays a role in the pathogenesis of central obesity and the appearance of the metabolic syndrome in humans. Increased expression of the 11βHSD1 gene is associated with metabolic abnormalities in obese women and that increased expression of this gene is suspected to contribute to the increased local conversion of cortisone to cortisol in adipose tissue of obese individuals (Engeli, et al., (2004) Obes. Res. 12: 9-17).

A new class of 11βHSD1 inhibitors, the arylsulfonamidothiazoles, was shown to improve hepatic insulin sensitivity and reduce blood glucose levels in hyperglycemic strains of mice (Barf et al. (2002) J. Med. Chem. 45: 3813-3815; Alberts et al. Endocrinology (2003) 144: 4755-4762). Additionally, it was recently reported that these selective inhibitors of 11βHSD1 can ameliorate severe hyperglycemia in genetically diabetic obese mice. Data using a structurally distinct series of compounds, the adamantyl triazoles (Hermanowski-Vosatka et al. (2005) J. Exp. Med. 202: 517-527), also indicates efficacy in rodent models of insulin resistance and diabetes, and further illustrates efficacy in a mouse model of atherosclerosis, perhaps suggesting local effects of corticosterone in the rodent vessel wall. Thus, 11βHSD1 is a promising pharmaceutical target for the treatment of the Metabolic Syndrome (Masuzaki, et al., (2003) Curr. Drug Targets Immune Endocr. Metabol. Disord. 3: 255-62).

A. Obesity and Metabolic Syndrome

As described above, multiple lines of evidence suggest that inhibition of 11βHSD1 activity can be effective in combating obesity and/or aspects of the metabolic syndrome cluster, including glucose intolerance, insulin resistance, hyperglycemia, hypertension, hyperlipidemia, and/or atherosclerosis/coronary heart disease. Glucocorticoids are known antagonists of insulin action, and reductions in local glucocorticoid levels by inhibition of intracellular cortisone to cortisol conversion should increase hepatic and/or peripheral insulin sensitivity and potentially reduce visceral adiposity. As described above, 11βHSD1 knockout mice are resistant to hyperglycemia, exhibit attenuated induction of key hepatic gluconeogenic enzymes, show markedly increased insulin sensitivity within adipose, and have an improved lipid profile. Additionally, these animals show resistance to high fat diet-induced obesity (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). In vivo pharmacology studies with multiple chemical scaffolds have confirmed the critical role for 11βHSD1 in regulating insulin resistance, glucose intolerance, dyslipidemia, hypertension, and atherosclerosis. Thus, inhibition of 11βHSD1 is predicted to have multiple beneficial effects in the liver, adipose, skeletal muscle, and heart, particularly related to alleviation of component(s) of the metabolic syndrome , obesity, and/or coronary heart disease.

B. Pancreatic Function

Glucocorticoids are known to inhibit the glucose-stimulated secretion of insulin from pancreatic beta-cells (Billaudel and Sutter (1979) Horm. Metab. Res. 11: 555-560). In both Cushing's syndrome and diabetic Zucker fa/fa rats, glucose-stimulated insulin secretion is markedly reduced (Ogawa et al. (1992) J. Clin. Invest. 90: 497-504). 11βHSD1 mRNA and activity has been reported in the pancreatic islet cells of ob/ob mice and inhibition of this activity with carbenoxolone, an 11βHSD1 inhibitor, improves glucose-stimulated insulin release (Davani et al. (2000) J. Biol. Chem. 275: 34841-34844). Thus, inhibition of 11βHSD1 is predicted to have beneficial effects on the pancreas, including the enhancement of glucose-stimulated insulin release and the potential for attenuating pancreatic beta-cell decompensation.

D. Cognition and Dementia

Mild cognitive impairment is a common feature of aging that may be ultimately related to the progression of dementia. In both aged animals and humans, inter-individual differences in general cognitive function have been linked to variability in the long-term exposure to glucocorticoids (Lupien et al. (1998) Nat. Neurosci. 1: 69-73). Further, dysregulation of the HPA axis resulting in chronic exposure to glucocorticoid excess in certain brain subregions has been proposed to contribute to the decline of cognitive function (McEwen and Sapolsky (1995) Curr. Opin. Neurobiol. 5: 205-216). 11βHSD1 is abundant in the brain, and is expressed in multiple subregions including the hippocampus, frontal cortex, and cerebellum (Sandeep et al. (2004) Proc. Natl. Acad. Sci. Early Edition: 1-6). Treatment of primary hippocampal cells with the 11βHSD1 inhibitor carbenoxolone protects the cells from glucocorticoid-mediated exacerbation of excitatory amino acid neurotoxicity (Rajan et al. (1996) J. Neurosci. 16: 65-70). Additionally, 11βHSD1-deficient mice are protected from glucocorticoid-associated hippocampal dysfunction that is associated with aging (Yau et al. (2001) Proc. Natl. Acad. Sci. 98: 4716-4721). In two randomized, double-blind, placebo-controlled crossover studies, administration of carbenoxolone improved verbal fluency and verbal memory (Sandeep et al. (2004) Proc. Natl. Acad. Sci. Early Edition: 1-6). Thus, inhibition of 11βHSD1 is predicted to reduce exposure to glucocorticoids in the brain and protect against deleterious glucocorticoid effects on neuronal function, including cognitive impairment, dementia, and/or depression.

E. Intra-Ocular Pressure

Glucocorticoids can be used topically and systemically for a wide range of conditions in clinical ophthalmology. One particular complication with these treatment regimens is corticosteroid-induced glaucoma. This pathology is characterized by a significant increase in intra-ocular pressure (IOP). In its most advanced and untreated form, IOP can lead to partial visual field loss and eventually blindness. IOP is produced by the relationship between aqueous humour production and drainage. Aqueous humour production occurs in the non-pigmented epithelial cells (NPE) and its drainage is through the cells of the trabecular meshwork. 11βHSD1 has been localized to NPE cells (Stokes et al. (2000) Invest. Ophthalmol. Vis. Sci. 41: 1629-1683; Rauz et al. (2001) Invest. Ophthalmol. Vis. Sci. 42: 2037-2042) and its function is likely relevant to the amplification of glucocorticoid activity within these cells. This notion has been confirmed by the observation that free cortisol concentration greatly exceeds that of cortisone in the aqueous humour (14:1 ratio). The functional significance of 11βHSD1 in the eye has been evaluated using the inhibitor carbenoxolone in healthy volunteers (Rauz et al. (2001) Invest. Ophthalmol. Vis. Sci. 42: 2037-2042). After seven days of carbenoxolone treatment, IOP was reduced by 18%. Thus, inhibition of 11βHSD1 in the eye is predicted to reduce local glucocorticoid concentrations and IOP, producing beneficial effects in the management of glaucoma and other visual disorders.

F. Hypertension

Adipocyte-derived hypertensive substances such as leptin and angiotensinogen have been proposed to be involved in the pathogenesis of obesity-related hypertension (Matsuzawa et al. (1999) Ann. N.Y. Acad. Sci. 892: 146-154; Wajchenberg (2000) Endocr. Rev. 21: 697-738). Leptin, which is secreted in excess in aP2-11βHSD1 transgenic mice (Masuzaki et al. (2003) J. Clinical Invest. 112: 83-90), can activate various sympathetic nervous system pathways, including those that regulate blood pressure (Matsuzawa et al. (1999) Ann. N.Y. Acad. Sci. 892: 146-154). Additionally, the renin-angiotensin system (RAS) has been shown to be a major determinant of blood pressure (Walker et al. (1979) Hypertension 1: 287-291). Angiotensinogen, which is produced in liver and adipose tissue, is the key substrate for renin and drives RAS activation. Plasma angiotensinogen levels are markedly elevated in aP2-11βHSD1 transgenic mice, as are angiotensin II and aldosterone (Masuzaki et al. (2003) J. Clinical Invest. 112: 83-90). These forces likely drive the elevated blood pressure observed in aP2-11βHSD1 transgenic mice. Treatment of these mice with low doses of an angiotensin II receptor antagonist abolishes this hypertension (Masuzaki et al. (2003) J. Clinical Invest. 112: 83-90). This data illustrates the importance of local glucocorticoid reactivation in adipose tissue and liver, and suggests that hypertension may be caused or exacerbated by 11βHSD1 activity. Thus, inhibition of 11βHSD1 and reduction in adipose and/or hepatic glucocorticoid levels is predicted to have beneficial effects on hypertension and hypertension-related cardiovascular disorders.

G. Bone Disease

Gluccorticoids can have adverse effects on skeletal tissues. Continued exposure to even moderate glucocorticoid doses can result in osteoporosis (Cannalis (1996) J. Clin. Endocrinol. Metab. 81: 3441-3447) and increased risk for fractures. Experiments in vitro confirm the deleterious effects of glucocorticoids on both bone-resorbing cells (also known as osteoclasts) and bone forming cells (osteoblasts). 11βHSD1 has been shown to be present in cultures of human primary osteoblasts as well as cells from adult bone, likely a mixture of osteoclasts and osteoblasts (Cooper et al. (2000) Bone 27: 375-381), and the 11βHSD1 inhibitor carbenoxolone has been shown to attenuate the negative effects of glucocorticoids on bone nodule formation (Bellows et al. (1998) Bone 23: 119-125). Thus, inhibition of 11βHSD1 is predicted to decrease the local glucocorticoid concentration within osteoblasts and osteoclasts, producing beneficial effects in various forms of bone disease, including osteoporosis.

Small molecule inhibitors of 11βHSD1 are currently being developed to treat or prevent 11βHSD1-related diseases such as those described above. For example, certain amide-based inhibitors are reported in WO 2004/089470, WO 2004/089896, WO 2004/056745, WO 2004/065351, and WO 2005/108359. Antagonists of 11βHSD1 have also been evaluated in human clinical trials (Kurukulasuriya, et al., (2003) Curr. Med. Chem. 10: 123-53).

In light of the experimental data indicating a role for 11βHSD1 in glucocorticoid-related disorders, metabolic syndrome, hypertension, obesity, insulin resistance, hyperglycemia, hyperlipidemia, type 2 diabetes, atherosclerosis, androgen excess (hirsutism, menstrual irregularity, hyperandrogenism) and polycystic ovary syndrome (PCOS), therapeutic agents aimed at augmentation or suppression of these metabolic pathways, by modulating glucocorticoid signal transduction at the level of 11βHSD1 are desirable.

Furthermore, because the MR binds to aldosterone (its natural ligand) and cortisol with equal affinities, compounds that are designed to interact with the active site of 11βHSD1 (which binds to cortisone/cortisol) may also interact with the MR and act as antagonists. Because the MR is implicated in heart failure, hypertension, and related pathologies including atherosclerosis, arteriosclerosis, coronary artery disease, thrombosis, angina, peripheral vascular disease, vascular wall damage, and stroke, MR antagonists are desirable and may also be useful in treating complex cardiovascular, renal, and inflammatory pathologies including disorders of lipid metabolism including dyslipidemia or hyperlipoproteinaemia, diabetic dyslipidemia, mixed dyslipidemia, hypercholesterolemia, hypertriglyceridemia, as well as those associated with type 1 diabetes, type 2 diabetes, obesity, metabolic syndrome, and insulin resistance, and general aldosterone-related target-organ damage.

As evidenced herein, there is a continuing need for new and improved drugs that target 11βHSD1. The compounds, compositions and methods therein help meet this and other needs.

### SUMMARY OF THE INVENTION

The present invention provides, inter alia, compounds of Formula I or Ia: 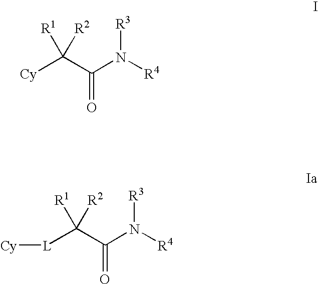
or pharmaceutically acceptable salts or prodrugs thereof, wherein constituent members are defined herein.

The present invention further provides compounds of Formula II, III, IV, Va, Vb, VI, VII, VIII, or IX: 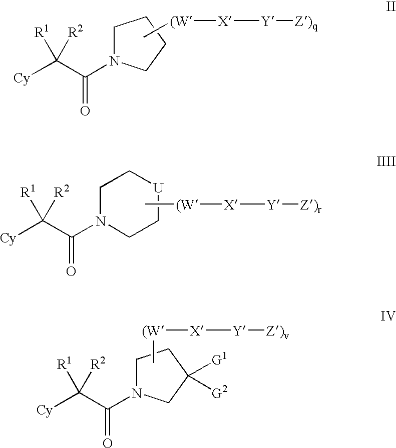 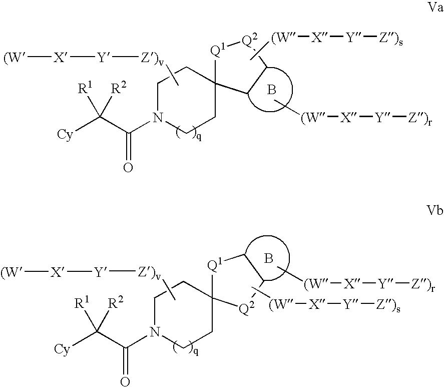 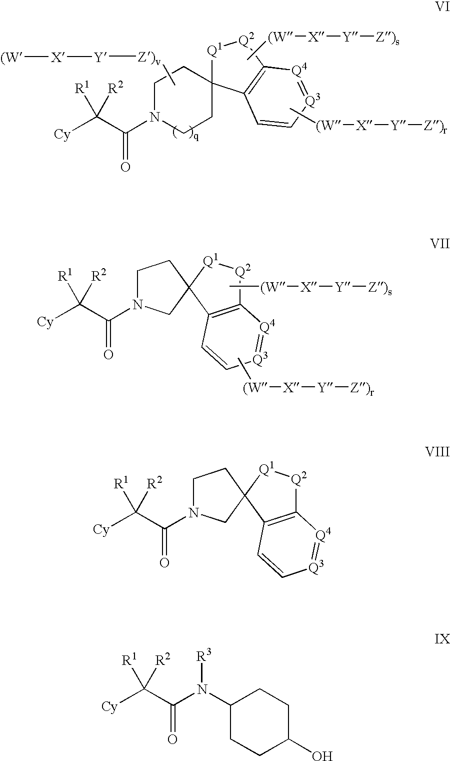
or pharmaceutically acceptable salts or prodrugs thereof, wherein constituent members are defined herein.

The present invention further provides methods of modulating 11βHSD1 by contacting 11βHSD1 with a compound of the invention.

The present invention further provides methods of inhibiting 11βHSD1 by contacting 11βHSD1 with a compound of the invention.

The present invention further provides methods of inhibiting the conversion of cortisone to cortisol in a cell by contacting the cell with a compound of the invention.

The present invention further provides methods of inhibiting the production of cortisol in a cell by contacting the cell with a compound of the invention.

The present invention further provides methods of treating diseases associated with activity or expression of 11βHSD1.

### DETAILED DESCRIPTION

The present invention provides, inter alia, a compound of of Formula I or Ia: 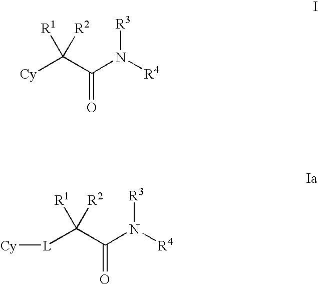
or a pharmaceutically acceptable salt or prodrug thereof, wherein:

Cy is aryl, heteroaryl, cycloalkyl or heterocycloalkyl, each optionally substituted by 1, 2, 3, 4 or 5 —W—X—Y-Z;

R¹ is H, F, CN, OR⁵, SR⁵, C₁₋₆ alkyl, C₁₋₆ haloalkyl, C₂₋₁₂ alkoxyalkyl, C₂₋₁₂ haloalkoxyalkyl, cylcoalkyl, heterocycloalkyl, cycloalkylalkyl or heterocycloalkylalkyl;

R² is H, F, CN, C₁₋₆ alkyl, C₁₋₆ haloalkyl, C₂₋₁₂ alkoxyalkyl, C₂₋₁₂ haloalkoxyalkyl, cylcoalkyl, heterocycloalkyl, cycloalkylalkyl or heterocycloalkylalkyl;

R³ is H, C₁₋₆alkyl, cycloalkyl or heterocycloalkyl, wherein each of the C₁₋₆alkyl, cycloalkyl, and heterocycloalkyl is optionally substituted by 1, 2, 3, 4 or 5 —W′—X′—Y′-Z′;

R⁴ is C₁₋₆ alkyl, aryl, cycloalkyl, heteroaryl or heterocycloalkyl, each optionally substituted by 1, 2, 3, 4 or 5 —W′—X′—Y′-Z′;

or R³ and R⁴ together with the N atom to which they are attached form a 4-20 membered heterocycloalkyl group optionally substituted by 1, 2, 3, 4 or 5 —W′—X′—Y′-Z′;

each R⁵ is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl and heterocycloalkylalkyl is optionally substituted by 1, 2, 3, 4 or 5 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};

L is SO₂, (CR⁶R⁷)ₙ₁O(CR⁶R⁷)ₙ₂, (CR⁶R⁷)ₙ₁S(CR⁶R⁷)ₙ₂, or (CR⁶R⁷)ₙ₃

R⁶ and R⁷ are independently selected from H, halo, C₁₋₆ alkyl, C₁₋₆ haloalkyl, aryl, cycloalkyl, heteroaryl, heterocycloalkyl, CN, NO₂, OR^{a′}, SR^{a′}, C(O)R^{b′}, C(O)NR^{c′}R^{d′, C(O)OR}^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, and S(O)₂NR^{c′}R^{d′};

n1 is 0, 1, 2 or 3;

n2 is 0, 1, 2 or 3;

n3 is 1, 2, 3 or 4;

W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;

X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₈ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;

Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of the C₁₋₆ alkylenyl, C₂₋₆ alkenylenyl and C₂₋₆ alkynylenyl is optionally substituted by 1, 2 or 3 substituent independently selected from halo, OH, C₁₋₆ alkoxy, C₁₋₆ haloalkoxy, amino, C₁₋₆ alkylamino and C₂₋₈ dialkylamino;

Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};

wherein two —W—X—Y-Z attached to the same atom optionally form a 3-14 membered cycloalkylk or 3-14 membered heterocycloalkyl group optionally substituted by 1, 2 or 3 —W″—X″″—Y″-Z″;

wherein two —W′—X′—Y″-Z″ attached to the same atom optionally form a 3-14 membered cycloalkyl or 3-14 membered heterocycloalkyl group optionally substituted by 1, 2 or 3 —W″—X″—Y″-Z″;

wherein —W—X—Y-Z is other than H;

wherein —W′—X′—Y′-Z′ is other than H;

wherein —W″—X″—Y″-Z″ is other than H;

each R^{a} and R^{a′} is independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;

each R^{b} and R^{b′} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C_{1,6} alkyl, C_{1,6} haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c′} and R^{d′} together with the N atom to which they are attached form a 4-, 5-, 6- or 7-membered heterocycloalkyl group; and

R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl.

In some embodiments, when R² is C₁₋₆ alkyl or C₁₋₆ haloalkyl, then R¹ is other than C₁₋₆ alkyl or C₁₋₆ haloalkyl.

In some embodiments, Cy is aryl or heteroaryl, each optionally substituted by 1, 2, 3, 4 or 5 —W—X—Y-Z.

In some embodiments, Cy is aryl or heteroaryl, each optionally substituted with 1, 2, 3, 4 or 5 —W—X—Y-Z wherein W is O or absent, X is absent, and Y is absent.

In some embodiments, Cy is aryl optionally substituted by 1, 2, 3, 4 or 5 —W—X—Y-Z.

In some embodiments, Cy is phenyl or naphthyl, each optionally substituted with 1, 2, 3, 4 or 5 —W—X—Y-Z.

In some embodiments, Cy is phenyl or naphthyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₂₋₈ alkoxyalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, C₁₋₆ cyanoalkyl, C₁₋₆ hydroxyalkyl, C₂₋₈ alkoxyalkyl, CN, NO₂, OR^{a}, SR^{a}, C(O)NR^{c}R^{d}, NR^{c}C(O)R^{d} and COOR^{a}.

In some embodiments, Cy is heteroaryl optionally substituted by 1, 2, 3, 4 or 5 —W—X—Y-Z.

In some embodiments, Cy is pyridyl, pyrimidinyl, triazinyl, furanyl, thiazolyl, pyrazinyl, purinyl, quinazolinyl, quinolinyl, isoquinolinyl, pyrrolo[2,3-d]pyrimidinyl, or 1,3-benzothiazolyl, each optionally substituted by 1, 2, 3, 4 or 5 —W—X—Y-Z.

In some embodiments, Cy is pyridyl, pyrimidinyl, triazinyl, furanyl, thiazolyl, pyrazinyl, purinyl, quinazolinyl, quinolinyl, isoquinolinyl, pyrrolo[2,3-d]pyrimidinyl, or 1,3-benzothiazolyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₂₋₈ alkoxyalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, C₁₋₆ cyanoalkyl, C₁₋₆ hydroxyalkyl, C₂₋₈ alkoxyalkyl, CN, NO₂, OR^{a}, SR^{a}, C(O)NR^{c}R^{d}, NR^{c}C(O)R^{d} and COOR^{a}.

In some embodiments, Cy is cycloalkyl or heterocycloalkyl, each optionally substituted by 1, 2, 3, 4 or 5 —W—X—Y-Z.

In some embodiments, Cy is cycloalkyl or heterocycloalkyl, each optionally substituted by 1, 2, 3, 4 or 5 —W—X—Y-Z wherein W is O or absent, X is absent, and Y is absent.

In some embodiments, cyclopropyl, cyclobutyl, cyclopentyl, cyclohexyl, cyclocheptyl, adamantyl, aziridinyl, azetidinyl, pyrrolidine, piperidinyl, piperizinyl or morpholinyl, each optionally substituted by 1, 2, 3, 4 or 5 —W—X—Y-Z.

In some embodiments, Cy is cyclopropyl, cyclobutyl, cyclopentyl, cyclohexyl, cyclocheptyl, adamantyl, aziridinyl, azetidinyl, pyrrolidine, piperidinyl, piperizinyl or morpholinyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₂₋₈ alkoxyalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, C₁₋₆ cyanoalkyl, C₁₋₆ hydroxyalkyl, C₂₋₈ alkoxyalkyl, CN, NO₂, OR^{a}, SR^{a}, C(O)NR^{c}R^{d}, NR^{c}C(O)R^{d} and COOR^{a}.

In some embodiments, R¹ is H, OR⁵, SR⁵ or C₁₋₆ alkyl; and each R⁵ is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl.

In some embodiments, R¹ is H. In some embodiments, R¹ is OR⁵ or SR⁵. In some embodiments, R¹ is OR⁵. In some embodiments, R¹ is OR⁵ or SR⁵; and each R⁵is independently H or C₁₋₆ alkyl. In some embodiments, R¹ is hydroxy, methoxy, or methylthio.

In some embodiments, R² is H, C₁₋₆ alkyl or C₁₋₆ haloalkyl. In some embodiments, R² is methyl or ethyl. In some embodiments, R² is methyl. In some embodiments, R² is H.

In some embodiments, R³ is H, C₁₋₆ alkyl, C₁₋₆ haloalkyl, C₁₋₆ hydroxyalkyl, C₂₋₁₂ alkoxyalkyl. In some further embodiments, R³ is H or C₁₋₆ alkyl. In yet further embodiments, R³ is C₁₋₆ alkyl.

In some embodiments, R⁴ is C₁₋₆ alkyl, cycloalkyl or heterocycloalkyl, each optionally substituted by 1, 2, 3, 4 or 5 —W′—X′—Y′-Z′.

In some embodiments, R⁴ is cycloalkyl optionally substituted by 1, 2, 3, 4 or 5 —W′—X′—Y′-Z′.

In some embodiments, R⁴ is cyclopropyl, cyclobutyl, cyclopentyl, cyclohexyl, cyclocheptyl, or adamantyl, each optionally substituted by 1, 2, 3 or 4 substituents independently selected from halo, C₁₋₆ alkyl, C₁₋₆ haloalkyl, C₁₋₆ cyanoalkyl, C₁₋₆ hydroxyalkyl, C₂₋₈ alkoxyalkyl, CN, NO₂, OH, C₂₋₈ alkoxyalkoxy, and C₁₋₄ alkoxy.

In some embodiments, R⁴ is cyclopropyl, cyclobutyl, cyclopentyl, cyclohexyl, cyclocheptyl, or adamantyl, each optionally substituted by 1, 2, 3 or 4 substituents independently selected from C₁₋₆ hydroxyalkyl, C₂₋₈ alkoxyalkyl, OH, C₂₋₈ alkoxyalkoxy, and C₁₋₄ alkoxy.

In some embodiments, R⁴ is heterocycloalkyl optionally substituted by 1, 2, 3, 4 or 5 —W′—X′—Y′-Z′.

In some embodiments, R⁴ is oxetanyl, tetrahydrofuranyl, tetrahydropyranyl, oxepanyl or morpholinyl, each optionally substituted by 1, 2, 3 or 4 substituents independently selected from halo, C₁₋₆ alkyl, C₁₋₆ haloalkyl, C₁₋₆ cyanoalkyl, C₁₋₆ hydroxyalkyl, C₂₋₈ alkoxyalkyl, CN, NO₂, OH, C₂₋₈ alkoxyalkoxy, and C₁₋₄ alkoxy.

In some embodiments, R⁴ is tetrahydrofuranyl or tetrahydropyranyl, each optionally substituted by 1, 2, 3 or 4 substituents independently selected from C₁₋₆ hydroxyalkyl, C₂₋₈ alkoxyalkyl, OH, C₂₋₈ alkoxyalkoxy, and C₁₋₄ alkoxy.

In some embodiments, R³ is H, C₁₋₆ alkyl, cycloalkyl or heterocycloalkyl, wherein each of the C₁₋₆alkyl, cycloalkyl, and heterocycloalkyl is optionally substituted by 1, 2, 3, 4 or 5 —W′—X′—Y′-Z′; and R⁴ is C₁₋₆ alkyl, aryl, cycloalkyl, heteroaryl or heterocycloalkyl, each optionally substituted by 1, 2, 3, 4 or 5 —W′—X′—Y′-Z′.

In some embodiments, R³ is H or C₁₋₆ alkyl; and R⁴ is cycloalkyl or heterocycloalkyl, each optionally substituted by 1, 2, 3, 4 or 5 —W′—X′—Y′-Z′.

In some embodiments, R³ is C₁₋₆ alkyl; and R⁴ is cyclopropyl, cyclobutyl, cyclopentyl, cyclohexyl, cyclocheptyl, or adamantyl, each optionally substituted by 1, 2, 3 or 4 substituents independently selected from halo, C₁₋₆ alkyl, C₁₋₆ haloalkyl, C₁₋₆ cyanoalkyl, C₁₋₆ hydroxyalkyl, C₂₋₈ alkoxyalkyl, CN, NO₂, OH, C₂₋₈ alkoxyalkoxy, and C₁₋₄ alkoxy.

In some embodiments, R³ is C₁₋₆ alkyl; and R⁴ is oxetanyl, tetrahydrofuranyl, tetrahydropyranyl, oxepanyl or morpholinyl, each optionally substituted by 1, 2, 3 or 4 substituents independently selected from halo, C₁₋₆ alkyl, C₁₋₆ haloalkyl, C₁₋₆ cyanoalkyl, C₁₋₆ hydroxyalkyl, C₂₋₈ alkoxyalkyl, CN, NO₂, OH, C₂₋₈ alkoxyalkoxy, and C₁₋₄ alkoxy.

In some embodiments, R³ and R⁴ together with the N atom to which they are attached form a 5-14 membered heterocycloalkyl group optionally substituted by 1, 2, 3, or 4 —W′—X′—Y′-Z′.

In some embodiments, R³ and R⁴ together with the N atom to which they are attached form a 4-7 membered heterocycloalkyl group optionally substituted by 1, 2, 3, or 4 —W′—X′—Y′-Z′.

In some embodiments, R³ and R⁴ together with the N atom to which they are attached form a piperidinyl or pyrrolidinyl group optionally substituted by 1, 2, 3, or 4 —W′—X′—Y′-Z′.

In some embodiments, R³ and R⁴ together with the N atom to which they are attached form a piperidinyl or pyrrolidinyl group substituted by 2, 3, or 4 —W′—X′—Y′-Z′; wherein two —W′—X′—Y′-Z′ are attached to the same atom and optionally form a 3-20 membered cycloalkyl or heterocycloalkyl group optionally substituted by 1, 2 or 3 —W″—X″—Y″-Z″.

In some embodiments, each —W—X—Y-Z is independently selected from halo, cyano, C₁₋₆ cyanoalkyl, nitro, C₁₋₈ alkyl, C₂₋₈ alkenyl, C₁₋₈ haloalkyl, C₁₋₆ alkylthio, C₁₋₆ haloalkylthio, C₁₋₈ alkoxy, C₂₋₈ alkenyloxy, C₁₋₆ haloalkoxy, OH, (C₁₋₆ alkoxy)-C₁₋₆ alkyl, amino, C₁₋₆ alkylamino, C₂₋₈ dialkylamino, OC(O)NR^{c}R^{d}, NR^{c}C(O)R^{d}, NR^{c}C(O)OR^{a}, NR^{c}S(O)₂R^{d}, C(O)OR^{a}, C(O)R^{a}, C(O)NR^{a}NR^{c}R^{d}, S(O)₂R^{d}, SR^{d}, C(O)NR^{c}R^{d}, C(S)NR^{c}R^{d}, aryloxy, heteroaryloxy, cycloalkyloxy, cycloalkenyloxy, heterocycloalkyloxy, arylalkyloxy, heteroarylalkyloxy, cycloalkylalkyloxy, heterocycloalkylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl, heteroarylalkynyl, cycloalkylalkyl, and heterocycloalkylalkyl;

wherein each of the C₁₋₈ alkyl, C₂₋₈ alkenyl, C₁₋₈ haloalkyl, C₁₋₆ alkylthio, C₁₋₆ haloalkylthio, C₁₋₈ alkoxy, aryloxy, heteroaryloxy, arylalkyloxy, heteroarylalkyloxy, heteroaryloxyalkyl, aryloxy, heteroaryloxy, cycloalkyloxy, cycloalkenyloxy, heterocycloalkyloxy, arylalkyloxy, heteroarylalkyloxy, cycloalkylalkyloxy, heterocycloalkylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl, heteroarylalkynyl, cycloalkylalkyl, and heterocycloalkylalkyl is optionally substituted by 1, 2, or 3 halo, cyano, nitro, C₁₋₆ hydroxyalkyl, C₁₋₆ cyanoalkyl, aminoalkyl, dialkylaminoalkyl, C₁₋₆ alkyl, C₁₋₆ haloalkyl, C₁₋₆ cyanoalkyl, C₁₋₆ alkoxy, C₁₋₆ haloalkoxy, OH, OR^{a}, (C₁₋₆ alkoxy)-C₁₋₆ alkyl, amino, C₁₋₆ alkylamino, C₂₋₈ dialkylamino, C(O)NR^{c}R^{d}, C(O)OR^{a}, C(O)R^{a}, (cyclocalkylalkyl)-C(O)—, NR^{c}C(O)R^{d}, NR^{c}C(O)OR^{a}, NR^{c}S(O)₂R^{d}, C(S)NR^{c}R^{d}, S(O)₂R^{d}, SR^{d}, (C₁₋₆ alkyl)sulfonyl, arylsulfonyl, aryl optionally substituted by halo, heteroaryl, cycloalkylalkyl, cycloalkyl, or heterocycloalkyl.

In some embodiments, each —W—X—Y-Z is independently selected from halo, cyano, C₁₋₆ cyanoalkyl, nitro, C₁₋₈ alkyl, C₁₋₈ alkenyl, C₁₋₈ haloalkyl, C₁₋₈ alkoxy, C₁₋₆ haloalkoxy, OH, C₁₋₈ alkoxyalkyl, amino, C₁₋₆ alkylamino, C₂₋₈ dialkylamino, OC(O)NR^{c}R^{d}, NR^{c}C(O)R^{d}, NR^{c}C(O)OR^{a}, aryloxy, heteroaryloxy, arylalkyloxy, heteroarylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl, heteroarylalkynyl, cycloalkylalkyl, or heterocycloalkylalkyl;

wherein each of the C₁₋₈ alkyl, C₁₋₈ alkenyl, C₁₋₈ haloalkyl, C₁₋₈ alkoxy, aryloxy, heteroaryloxy, arylalkyloxy, heteroarylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl, heteroarylalkynyl, cycloalkylalkyl, and heterocycloalkylalkyl is optionally substituted by 1, 2, or 3 substituents independently selected from halo, cyano, nitro, C₁₋₆ hydroxyalkyl, C₁₋₆ cyanoalkyl, aminoalkyl, dialkylaminoalkyl, C₁₋₆ alkyl, C₁₋₆ haloalkyl, C₁₋₆ alkoxy, C₁₋₆ haloalkoxy, OH, C₁₋₈ alkoxyalkyl, amino, C₁₋₆ alkylamino, C₂₋₈ dialkylamino, C(O)NR^{c}R^{d}, C(O)OR^{a}, NR^{c}C(O)R^{d}, NR^{c}S(O)₂R^{d}, (C₁₋₆ alkyl)sulfonyl, arylsulfonyl, aryl, heteroaryl, cycloalkyl, and heterocycloalkyl.

In some embodiments, each —W—X—Y-Z is independently selected from halo, C₁₋₆ alkyl, C₁₋₆ alkoxy, C₁₋₆ haloalkyl, C₁₋₆ haloalkoxy, aryl and heteroaryl, wherein each of the aryl and heteroaryl is optionally substituted by 1, 2, or 3 substituents independently selected from halo, cyano, nitro, C₁₋₆ hydroxyalkyl, C₁₋₆ cyanoalkyl, aminoalkyl, dialkylaminoalkyl, C₁₋₆ alkyl, C₁₋₆ haloalkyl, C₁₋₆ alkoxy, C₁₋₆haloalkoxy, OH, C₂₋₁₂ alkoxyalkoxy, C₂₋₁₂ alkoxyalkyl, amino, C₁₋₆ alkylamino, C₂₋₈ dialkylamino, C(O)NR^{c}R^{d}, C(O)OR^{a}, NR^{c}C(O)R^{d}, NR^{c}S(O)₂R^{d}, (C₁₋₆ alkyl)sulfonyl, arylsulfonyl, aryl, heteroaryl, cycloalkyl, and heterocycloalkyl.

In some embodiments, each —W—X—Y-Z is independently selected from halo, cyano, C₁₋₆ cyanoalkyl, nitro, C₁₋₆ nitroalkyl, C₁₋₆ alkyl, C₁₋₆ haloalkyl, C₁₋₆ alkoxy, C₁₋₆ haloalkoxy, OH, (C₁₋₆ alkoxy)-C₁₋₆ alkyl, amino, C₁₋₆ alkylamino, C₂₋₈ dialkylamino, aryl, heteroaryl, cycloalkyl, heterocycloalkyl, arylalkyl, heteroarylalkyl, cycloalkylalkyl, and heterocycloalkylalky.

In some embodiments:

each —W′—X′—Y′-Z′ is independently selected from halo, OH, cyano, CHO, COOH, C(O)O—(C₁₋₆ alkyl), C(O)—(C₁₋₆ alkyl), SO₂—(C₁₋₆ alkyl), C₁₋₆ alkyl, C₁₋₆ alkoxy and -L-R⁷, wherein the C₁₋₆ alkyl or C₁₋₆ alkoxy is optionally substituted by 1, 2, 3, 4, or 5 substituents independently selected from halo, OH, COOH and C(O)O—(C₁₋₆ alkyl);

L is absent, O, CH₂, NHSO₂, or N[C(O)—(C₁₋₆ alkyl)]; and

R⁷ is aryl or heteroaryl, each optionally substituted by 1, 2, or 3 substituents independently selected from halo, OH, cyano, CHO, COOH, C(O)O—(C₁₋₆ alkyl), C(O)—(C₁₋₆ alkyl), SO₂—(C₁₋₆ alkyl), SO₂—NH(C₁₋₆ alkyl), C₁₋₆ alkyl, C₁₋₆ alkoxy, C₁₋₆ haoalkyl, C₁₋₆ hydroxyalkyl, aryl, heteroaryl and aryloxy.

In some embodiments, each —W′—X′—Y′-Z′ is indepently halo; C₁₋₆ alkyl; C₁₋₆ haloalkyl; OH; C₁₋₆ alkoxy; C₁₋₆ haloalkoxy; C₂₋₁₂ alkoxyalkoxy; C₁₋₆ hydroxyalkyl; C₂₋₁₂ alkoxyalkyl; aryl, heteroaryl; aryl substituted by halo, C₁₋₆ alkyl, C₁₋₆ alkoxy, aryl, heteroaryl, or aryloxy; or heteroaryl substituted by halo, C₁₋₆ alkyl, C₁₋₆ alkoxy, aryl, or heteroaryl.

In some embodiments, two —W′—X′—Y′-Z′ are attached to the same atom and optionally form a 3-20 membered cycloalkyl or heterocycloalkyl group optionally substituted by 1, 2 or 3 —W″—X″—Y″-Z″.

In some embodiments, each —W″—X″—Y″-Z″ is indepently halo, cyano, C₁₋₆ cyanoalkyl, nitro, C₁₋₆ nitroalkyl, C₁₋₆ alkyl, C₁₋₆ haloalkyl, C₁₋₆ alkoxy, C₁₋₆ haloalkoxy, OH, (C₁₋₆ alkoxy)-C₁₋₆ alkyl, amino, C₁₋₆ alkylamino, C₂₋₈ dialkylamino, aryl, heteroaryl, cycloalkyl, heterocycloalkyl, arylalkyl, heteroarylalkyl, cycloalkylalkyl, or heterocycloalkylalkyl.

In some embodiments, the compounds of the invention have Formula I.

In some embodiments, the compounds of the invention have Formula Ia and L is SO₂.

In some embodiments, the compounds of the invention have Formula Ia and L is (CR⁶R⁷)ₙ₁O(CR⁶R⁷)ₙ₂.

In some embodiments, the compounds of the invention have Formula Ia and L is OCH₂.

In some embodiments, the compounds of the invention have Formula Ia and L is (CR⁶R⁷)ₙ₁S(CR⁶R⁷)ₙ₂.

In some embodiments, the compounds of the invention have Formula Ia and L is S or SCH₂.

In some embodiments, the compounds of the invention have Formula Ia and L is S.

In some embodiments, the compounds of the invention have Formula Ia and L is SCH₂.

In some embodiments, the compounds of the invention have Formula Ia and L is (CR⁶R⁷)ₙ₃.

In some embodiments, the compounds of the invention have Formula Ia and L is —CH₂—, —CH₂CH₂— or —CH₂CH₂CH₂—.

In some embodiments, the compounds of the invention have Formula II: 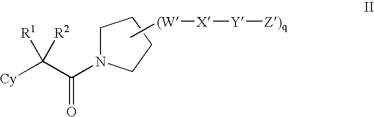
wherein:

Cy is aryl, heteroaryl, cycloalkyl or heterocycloalkyl, each optionally substituted by 1, 2, 3, 4 or 5 —W—X—Y-Z;

R¹ is H, OR⁵ or SR⁵;

R² is H, C₁₋₆ alkyl or C₁₋₆ haloalkyl;

each R⁵ is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl and heterocycloalkylalkyl is optionally substituted by 1, 2, 3, 4 or 5 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};

W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;

X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₈ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;

Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;

Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alknyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{d}, S(O)₂R^{b}, and S(O)₂NR^{c}R^{d};

wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;

wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;

wherein —W—X—Y-Z is other than H;

wherein —W′—X′—Y′-Z′ is other than H;

wherein —W″—X″—Y″-Z″ is other than H;

each R^{a} is independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;

each R^{b} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl; and

q is 0, 1, 2, 3 or 4.

In some embodiments, the compounds of the invention have Formula III: 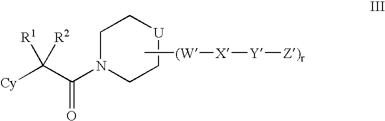
or pharmaceutically acceptable salt or prodrug thereof, wherein:

Cy is aryl, heteroaryl, cycloalkyl or heterocycloalkyl, each optionally substituted by 1, 2, 3, 4 or 5 —W—X—Y-Z;

U is NH, CH₂ or O;

R¹ is H, OR⁵ or SR⁵;

R² is H, C₁₋₆ alkyl or C₁₋₆ haloalkyl;

each R⁵ is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl and heterocycloalkylalkyl is optionally substituted by 1, 2, 3, 4 or 5 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};

W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;

X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₈ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;

Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO_{2,} SONR^{e}, and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;

Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR₂, SR^{a}, C(O)R^{b}, C(O)NR^{c}R^{d}, C(O)OR^{a}, OC(O)R^{b}, OC(O)NR^{c}R^{d}, NC^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};

wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;

wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;

wherein —W—X—Y-Z is other than H;

wherein —W′—X′—Y′-Z′ is other than H;

wherein —W″—X″—Y″-Z″ is other than H;

each R^{a} is independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;

each R^{b} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl; and

r is 0, 1, 2, 3 or 4.

In some embodiments, the compounds of the invention have Formula IV: 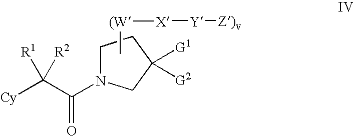
wherein:

Cy is aryl, heteroaryl, cycloalkyl or heterocycloalkyl, each optionally substituted by 1, 2, 3, 4 or 5 —W—X—Y-Z;

R¹ is H, OR⁵ or SR⁵;

R² is H, C₁₋₆ alkyl or C₁₋₆ haloalkyl;

each R⁵ is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl and heterocycloalkylalkyl is optionally substituted by 1, 2, 3, 4 or 5 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};

G¹ and G² together with the carbon atom to which they are attached form a 3-20 membered cycloalkyl or heterocycloalkyl group optional substituted by 1, 2 or 3 —W″—X″—Y″-Z″.

W, W′ and W″ are independently selected from absent, C₁₋₆ alkylenyl, C₂₋₆ alkenylenyl, C₂₋₆ alkynylenyl, O, S, NR^{c}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;

X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₈ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;

Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;

Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};

wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;

wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;

wherein —W—X—Y-Z is other than H;

wherein —W′—X′—Y′-Z′ is other than H;

wherein —W″—X″—Y″-Z″ is other than H;

each R^{a} is independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;

each R^{b} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl; and

v is 0, 1 or 2.

In some embodiments, the compounds of the invention have Formula Va or Vb: 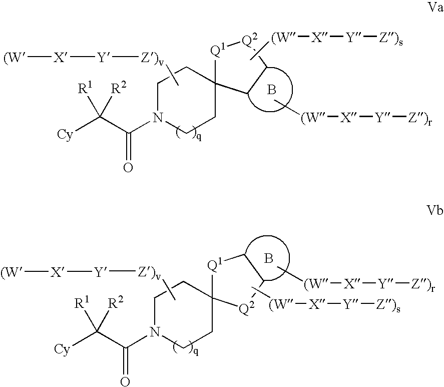
wherein:

ring B is a fused 5 or 6-membered aryl or heteroaryl group;

Q¹ is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;

Q² is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;

Cy is aryl, heteroaryl, cycloalkyl or heterocycloalkyl, each optionally substituted by 1, 2, 3, 4 or 5 —W—X—Y-Z;

R¹ is H, OR⁵ or SR⁵;

R² is H, C₁₋₆ alkyl or C₁₋₆ haloalkyl;

each R⁵ is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl and heterocycloalkylalkyl is optionally substituted by 1, 2, 3, 4 or 5 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};

W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;

X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₈ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;

Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;

Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, N^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};

wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;

wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;

wherein —W—X—Y-Z is other than H;

wherein —W′—X′—Y′-Z′ is other than H;

wherein —W″—X″—Y″-Z″ is other than H;

each R^{a} is independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;

each R^{b} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl; and

q is 0 or 1;

v is 0, 1 or 2;

r is 0, 1 or 2;

s is 0, 1 or 2; and

the sum of r and s is 0, 1 or 2.

In some embodiments, the compounds of the invention have Formula VI: 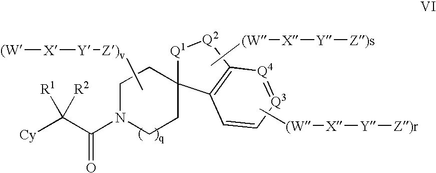
wherein:

Q¹ is O, S, NH, CH₂, CO, CS, SO, SO₂, OCH₂, SCH₂, NHCH₂, CH₂CH₂, CH═CH, COCH₂CONH, COO, SOCH₂, SONH, SO₂CH₂, or SO₂NH;

Q² is O, S, NH, CH₂, CO, CS, SO, SO₂, OCH₂, SCH₂, NHCH₂, CH₂CH₂, CH═CH, COCH₂CONH, COO, SOCH₂, SONH, SO₂CH₂, or SO₂NH;

Q³ and Q⁴ are independently selected from CH and N.

q is 0 or 1;

v is 0, 1 or 2;

r is 0, 1 or 2;

s is 0, 1 or 2;

the sum of r and s is 0, 1 or 2; and

Cy, R¹, R², W′, W″, X′, X″, Y′, Y″, Z′ and Z″ have any of the meanings defined hereinwith.

In some embodiments, the compounds of the invention have Formula VII: 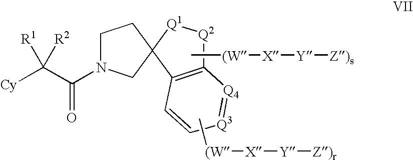
wherein:

Q¹ is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;

Q² is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;

Q³ and Q⁴ are independently selected from CH and N.

r is 0, 1 or 2;

s is 0, 1 or 2;

the sum of r and s is 0, 1 or 2; and

Cy, R¹, R², W′, W″, X′, X″, Y′, Y″, Z′ and Z″ have any of the meanings defined hereinwith.

In some embodiments, the compounds of the invention have Formula VIII: 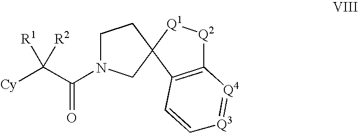
wherein:

Q¹ is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;

Q² is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;

Q³ and Q⁴ are independently selected from CH and N; and

Cy, R¹, R², W′, W″, X′, X″, Y′, Y″, Z′ and Z″ have any of the meanings defined hereinwith.

In some embodiments, at least one of R¹ and R² is other than H.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

For compounds of the invention in which a variable appears more than once, each variable can be a different moiety selected from the Markush group defining the variable. For example, where a structure is described having two R groups that are simultaneously present on the same compound; the two R groups can represent different moieties selected from the Markush group defined for R. In another example, when an optionally multiple substituent is designated in the form: 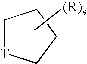
then it is understood that substituent R can occur s number of times on the ring, and R can be a different moiety at each occurrence. Further, in the above example, should the variable T be defined to include hydrogens, such as when T is said to be CH₂, NH, etc., any floating substituent such as R in the above example, can replace a hydrogen of the T variable as well as a hydrogen in any other non-variable component of the ring.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms. The term “alkylene” refers to a divalent alkyl linking group.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “haloalkyl” refers to an alkyl group having one or more halogen substituents. Example haloalkyl groups include CF₃, C₂F₅, CHF₂, CCl₃, CHCl₂, C₂Cl₅, CH₂CF₃, and the like.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic cyclic hydrocarbons including cyclized alkyl, alkenyl, and alkynyl groups. Cycloalkyl groups can include mono- or polycyclic (e.g., having 2, 3 or 4 fused rings) ring systems as well as spiro ring systems. Ring-forming carbon atoms of a cycloalkyl group can be optionally substituted by oxo or sulfido. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to non-aromatic heterocycles where one or more of the ring-forming carbon atoms is a heteroatom such as an O, N, or S atom. Hetercycloalkyl groups can be mono or polycyclic (e.g., both fused and spiro systems). Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used herein, “haloalkoxy” refers to an —O-haloalkyl group. An example haloalkoxy group is OCF₃.

As used herein, “alkoxyalkyl” refers to an alkyl group substituted by an alkoxy group. One example of alkoxyalkyl is —CH₂—OCH₃.

As used herein, “cyanoalkyl” refers to an alkyl group substituted by a cyano group (CN). One example of cyanoalkyl is —CH₂—CN.

As used herein, “alkoxyalkoxy” refers to an alkoxy group substituted by an alkoxy group. One example of alkoxyalkoxy is —OCH₂CH₂—OCH₃.

As used herein, “arylalkyl” refers to alkyl substituted by aryl and “cycloalkylalkyl” refers to alkyl substituted by cycloalkyl. An example arylalkyl group is benzyl. As used herein, “arylalkenyl” refers to alkenyl substituted by aryl and “arylalkynyl” refers to alkynyl substituted by aryl.

As used herein, “heteroarylalkyl” refers to an alkyl group substituted by a heteroaryl group, and “heterocycloalkylalkyl” refers to alkyl substituted by heterocycloalkyl. As used herein, “heteroarylalkenyl” refers to alkenyl substituted by heteroaryl and “heteroarylalkynyl” refers to alkynyl substituted by heteroaryl.

As used herein, “amino” refers to NH₂.

As used herein, “alkylamino” refers to an amino group substituted by an alkyl group.

As used herein, “dialkylamino” refers to an amino group substituted by two alkyl groups.

As used herein, “dialkylaminocarbonyl” refers to a carbonyl group substituted by a dialkylamino group.

As used herein, “dialkylaminocarbonylalkyloxy” refers to an alkyloxy (alkoxy) group substituted by a carbonyl group which in turn is substituted by a dialkylamino group.

As used herein, “cycloalkylcarbonyl(alkyl)amino” refers to an alkylamino group substituted by a carbonyl group (on the N atom of the alkylamino group) which in turn is substituted by a cycloalkyl group. The term “cycloalkylcarbonylamino” refers to an amino group substituted by a carbonyl group (on the N atom of the amino group) which in turn is substituted by a cycloalkyl group. The term “cycloalkylalkylcarbonylamino” refers to an amino group substituted by a carbonyl group (on the N atom of the amino group) which in turn is substituted by a cycloalkylalkyl group.

As used herein, “alkoxycarbonyl(alkyl)amino” refers to an alkylamino group substituted by an alkoxycarbonyl group on the N atom of the alkylamino group. The term “alkoxycarbonylamino” refers to an amino group substituted by an alkoxycarbonyl group on the N atom of the amino group.

As used herein “alkoxycarbonyl” refers to a carbonyl group [—C(O)—] substituted by an alkoxy group.

As used herein, “alkylsulfonyl” refers to a sulfonyl group [—S(O)₂—] substituted by an alkyl group. The term “alkylsulfonylamino” refers to an amino group substituted by an alkylsulfonyl group.

As used herein, “arylsulfonyl” refers to a sulfonyl group [—S(O)₂—] substituted by an aryl group, i.e., —S(O)₂-aryl.

As used herein, “dialkylaminosulfonyl” refers to a sulfonyl group substituted by dialkylamino.

As used herein, “arylalkyloxy” refers to —O-arylalkly. An example of an arylalkyloxy group is benzyloxy.

As used herein, “cycloalkyloxy” refers to —O-cycloalkyl. An example of a cycloalkyloxy group is cyclopenyloxyl.

As used herein, “heterocycloalkyloxy” refers to —O-heterocycloalkyl.

As used herein, “aryloxy” refers to —O-aryl. An example of aryloxy is phenoxy. The term “aryloxyalkyl” refers to an alkyl group substituted by an aryloxy group.

As used herein, “heteroaryloxy” refers to —O-heteroaryl. An example is pyridyloxy. The term “heteroaryloxyalkyl” refers to an alkyl group substituted by a heteroaryloxy group.

As used herein, “acylamino” refers to an amino group substituted by an alkylcarbonyl (acyl) group. The term “acyl(alkyl)amino” refers to an amino group substituted by an alkylcarbonyl (acyl) group and an alkyl group.

As used herein, “alkylcarbonyl” refers to a carbonyl group substituted by an alkyl group.

As used herein, “cycloalkylaminocarbonyl” refers to a carbonyl group substituted by an amino group which in turn is substituted by a cycloalkyl group.

As used herein, “aminocarbonyl” refers to a carbonyl group substituted by an amino group (i.e., CONH₂).

As used herein, “hydroxyalkyl” refers to an alkyl group substituted by a hydroxyl group. An example is —CH₂OH.

As used herein, “alkylthio” refers to —S-alkyl, and “methylthio” refers to —S—CH₃.

As used herein, “alkylcarbonyloxy” refers to an oxy group substituted by a carbonyl group which in turn is substituted by an alkyl group [i.e., —O—C(O)-(alkyl)].

As used herein, the terms “substitute” or “substitution” refer to replacing a hydrogen with a non-hydrogen moiety.

As used used herein, the term “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valency of the designated atom or moiety is not exceeded, and that the substitution results in a stable compound. For example, if a methyl group (i.e., CH₃) is optionally substituted, then 3 hydrogens on the carbon atom can be replaced with substituent groups.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H- 1,2,4-triazole, 1H- and 2H-isoindole, and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention are intended to include compounds with stable structures. As used herein, “stable compound” and “stable structure” are meant to indicate a compound that is sufficiently robust to survive isolation to a useful degree of purity from a reaction mixture, and formulation into an efficacious therapeutic agent.

The term, “compound,” as used herein is meant to include all stereoisomers, geometric iosomers, tautomers, and isotopes of the structures depicted.

All compounds, and pharmaceuticaly acceptable salts thereof, are also meant to include solvated or hydrated forms.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Synthesis

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C NMR), infrared spectroscopy (IR), spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of the invention can be prepared, for example, using the reaction pathways and techniques as described below.

A series of carboxamides of formula 1-2 can be prepared by the method outlined in Scheme 1. A carboxylic acid 1-1 can be coupled to an appropriate amine HNR³R⁴ in the presence of a suitable peptide coupling reagent and in the presence of a suitable base such as a tertiary amine [e.g., triethylamine (Et₃N or TEA), diisopropylethylamine (iPr₂NEt or DIPEA), pyridine, and/or dimethylaminopyridine (DMAP)] to provide the desired product 1-2. Some non-limiting examples of suitable coupling reagents include 1,1′-carbonyl-diimidazole, N-(dimethylaminopropyl)-N′-ethyl carbodiimde, benzotriazol-1-yloxy-tris(dimethylamino)phosphonium hexafluoro-phosphate, and propanephosphonic anhydride.

The coupling reaction can be carried out in a suitable organic solvent. Some suitable organic solvent include polar organic solvent such as an alcohol (e.g., methanol, ethanol or isopropanol), or tetrahydrofuran (THF). Some suitable organic solvent include aprotic solvent. Some suitable organic solvent include polar aprotic organic solvent such as N,N-dimethylformamide (DMF), tetrahydrofuran (THF), dimethyl sulfoxide (DMSO) or methylene chloride.

Alternatively, the acid 1-1 can be converted to a more reactive acid derivative such as an acid chloride, ester, or a (mixed) anhydride, and the acid derivative can be optionally separated. The acid derivative can further be reacted with a desired amine HNR³R⁴ in the presence of a suitable base such as triethylamine or pyridine to generate the corresponding amide 1-2.

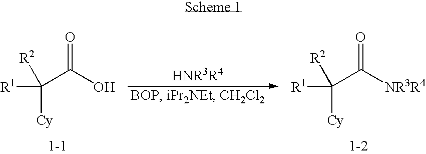

A series of carboxylic acids of formula 2-3 (wherein R² is alkyl, arylalkyl or the like) can be prepared by the method outlined in Scheme 2. In the presence of a suitable base such as sodium hydride and in a suitable solvent such as DMSO, mono-alkylation of an alpha-substituted methyl ester 2-1 with an alkyl bromide or alkyl iodide (R²Br or R²I) provides a mono-alkylated carboxylate 2-2. Basic hydrolysis of the carboxylate 2-2 gives the corresponding carboxylic acid 2-3.

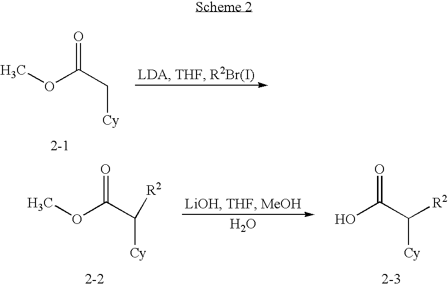

A series of carboxylic acids of formula 3-3 (wherein R¹ and R² can be alkyl, arylalkyl or the like) can be prepared by the method outlined in Scheme 3. An alpha-substituted acetonitrile 3-1 can be treated with a suitable base such as sodium hydride and and alkyl bromide or alkyl iodide (R²Br or R²I) in a suitable solvent such as DMF to provide the di-substituted carbonitrile 3-2. Basic hydrolysis of the carbonitrile 3-2 affords the corresponding carboxylic acid 3-3.

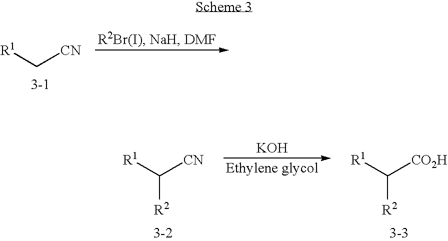

A series of acids of formula 4-6 (wherein R⁵ is alkyl, arylalkyl or the like) can be synthesized by method shown in Scheme 4. An acid chloride 4-1 can be reacted with a cyanide salt (e.g. KCN) to yield the compound 4-2. The cyano group of the compound 4-2 can be hydrolyzed under acidic condition (such as in the presence of hydrochloric acid) to afford the corresponding carboxylic acid and the carboxylic acid can then undergo esterification (such as in the presence of an alcohol and HCl) to generate an alpha-ketone ester 4-3. When subjected to a suitable reducing condition, such as ruthenium or rhodium catalyzed hydrogenation, the ketone 4-3 can be reduced to an alcohol 4-4. The alcohol 4-4 can then be alkylated (such as with R⁵Br) and then hydrolyzed to provide the corresponding acid 4-6.

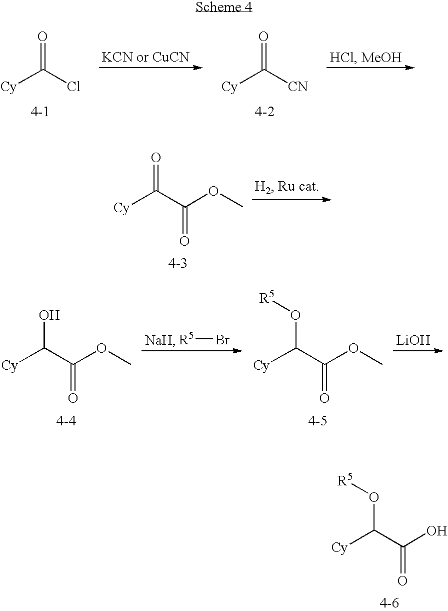

Alternatively, an acid with formula 5-4 can be prepared from an aldehyde 5-1 as illustrated in Scheme 5. An aldehyde 5-1 can be treated with sodium cyanide or chloroform in the presence of a base (e.g. sodium hydroxide) and a phase transfer reagent (e.g. a quaternary ammonium salt) to afford an alpha-hydroxy nitrile intermediate or an alpha-hydroxy trichloromethane intermediate respectively. Both of the intermediate can be hydrolyzed in the presence of an acid or base to furnish the alpha-hydroxy acid 5-2. The alpha-hydroxy acid 5-2 can then be alkylated with R⁵Br or R⁵I (wherein R⁵ is alkyl, arylalkyl or the like), and the alkylated 5-3 is further hydrolyzed to afford the acid 5-4.

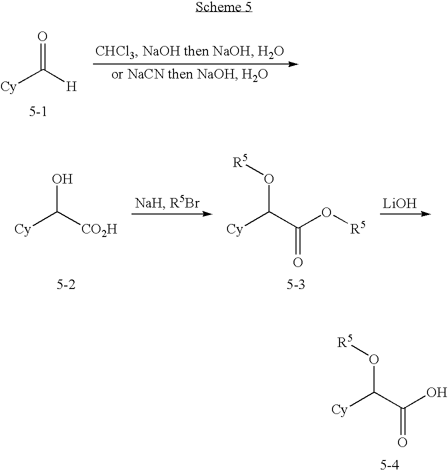

A series of acids of formula 6-4 can be prepared according to Scheme 6. Reaction of an alpha-ketone ester 6-1 with a suitable Grignard reagent R²MgBr (wherein R² is alkyl, arylalkyl, cycloalkyl or the like) or an alkyl lithium reagent R²Li gives compound 6-2. The compound 6-2 can be alkylated using an alkyl halide R⁵X¹ (wherein R⁵ is alkyl, arylalkyl or the like; and X¹ is chloride, bromide or iodide) and in the presence of a suitable base such as sodium hydride to generate an ether-ester 6-3. The ether-ester 6-3 can be further hydrolyzed under a suitable condition (e.g., in the presence of LiOH) to give the acid 6-4.

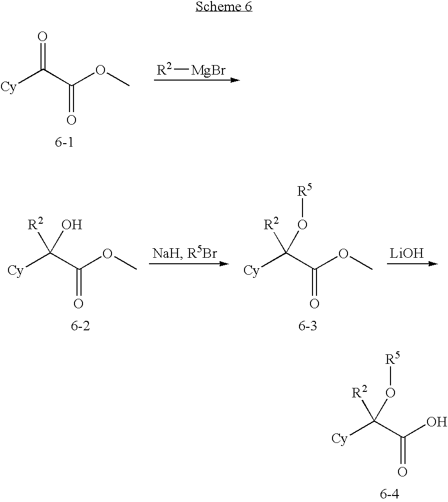

Primary amines of formula 7-2 (wherein R^{x} is a suitable substituent such as alkyl, haloalkyl, cycloalkyl or aryl; U is, e.g., CH₂, O, NMe, NBoc, etc.; n, e.g., is 1 or 2, m is, e.g., 0, 1 or 2; t1 is 0, 1, 2, etc.) can be prepared from an appropriate cyclic ketone 7-1 under a variety of protocols, one of which is shown in Scheme 7. The ketone 7-1 can undergoe reductive amination with ammonium formamide to afford the amine 7-2.

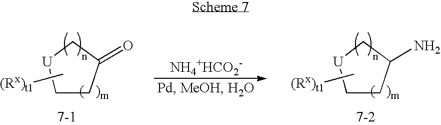

As shown in Scheme 8, alternatively, primary amines 8-4 (same as 7-2 in Scheme 7) can be prepared from the corresponding alcohols 8-1 via mesylation, followed by conversion of the mesylates 8-2 to the corresponding azides 8-3, which upon reduction yield the desired primary amines 8-4.

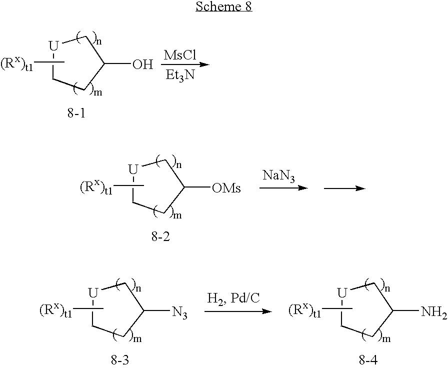

According to Scheme 9, a secondary amine of formula 9-2 (wherein R^{x} is a suitable substituent such as alkyl, haloalkyl, cycloalkyl or aryl; U is, e.g., CH₂, O, NMe, NBoc, etc.; n, e.g., is 1 or 2, m is, e.g., 0, 1 or 2; t1 is 0, 1, 2, etc.) can be prepared from reaction of an appropriate cyclic amine 9-1 with a suitable acid chloride R′COCl (wherein R′ is, e.g., alkyl, cycloalkyl, aryl, heteroaryl, heterocycloalkyl, arylalkyl, or the like) followed by reduction of the corresponding amide intermediate.

A secondary amine with formula 9-4 can be prepared from reductive amination of a ketone 9-3 with a suitable amine R′NH₂, as described in Scheme 9.

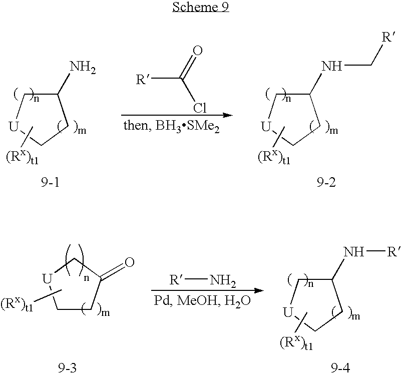

A series of 3-substituted pyrrolidine 10-3 and 10-5 can be prepared by the method outlined in Scheme 10 (wherein R′ is, e.g., —W′—X′—Y′-Z′). Compound 10-1 can be treated with an organolithium R′Li or a Grinard reagent R′MgBr to provide an alcohol 10-2. The Boc protecting group of 10-2 can be removed by treatment with TFA to give 3-substituted pyrrolidine 10-3. Alternatively, alcohol 10-2 can be treated with HCl to provide an alkene 10-4, upon hydrogenation which gives a 3-substituted pyrrolidine 10-5.

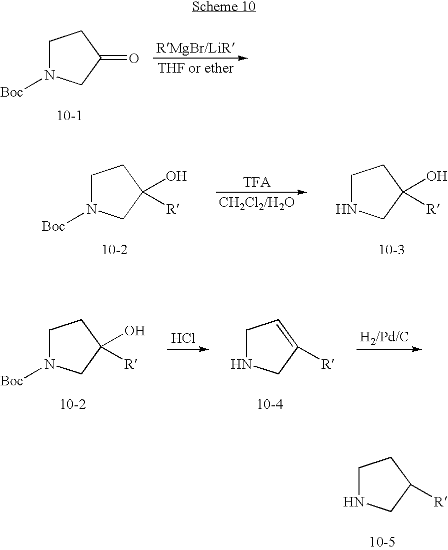

A series of 3-substituted pyrrolidines 29 can be prepared by the method outlined in Scheme 11 (wherein Ar is an aromatic moiety, i.e., an aryl or heteroaryl group which is optionally substituted by one or more substitutents such as halo, alkyl, etc.). A sequence of a Pd catalyzed coupling reaction of an alkene 11-1 with an optionally substituted aryl bromide or an optionally substituted heteroaryl bromide ArBr, followed by hydrogenation provides the desired 3-substituted pyrrolindine 11-2.

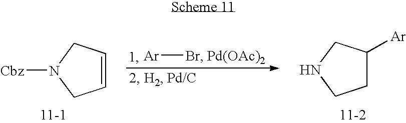

A series of 3-hydroxyl-4-substituted pyrrolidines 12-3 can be prepared by the method outlined in Scheme 12 (wherein Ar is an aromatic moiety, i.e., an aryl or heteroaryl group which is optionally substituted by one or more substitutents such as halo, alkyl, etc.). The alkene 12-1 can be reacted with mCPBA to provide the corresponding epoxide, which upon treatment with an organolithium ArLi or a Grignard reagent ArMgBr in the presence of a Lewis acid such as Al(Me)₃ gives an alcohol 12-2. Hydrogenolysis of the compound 12-2 provides the desired amine 12-3.

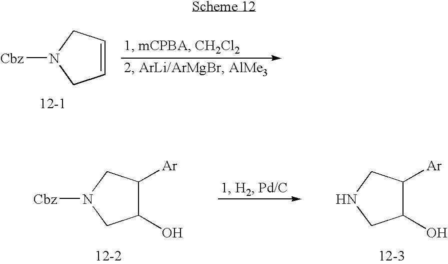

A series of 3,3-di-substituted pyrrolidines or piperidines 13-4 can be prepared by the method outlined in Scheme 13 (Ar is, for example, optionally substituted aryl or heteroaryl; n is 1 or 2 and m is 1 or 2). An ketone 13-1 can be treated with an appropriate Wittig reagent to provide an olefinic compound 13-2. Reaction of the olefinic compound 13-2 with an organocuprate Ar₂CuLi provides the corresponding 1,4 addition product 13-3. The Cbz protecting group of the compound 13-3 can be cleaved by hydrogenation to provide the desired 3,3-di-substituted pyrrolidine or 3,3-di-substituted piperidine 13-4.

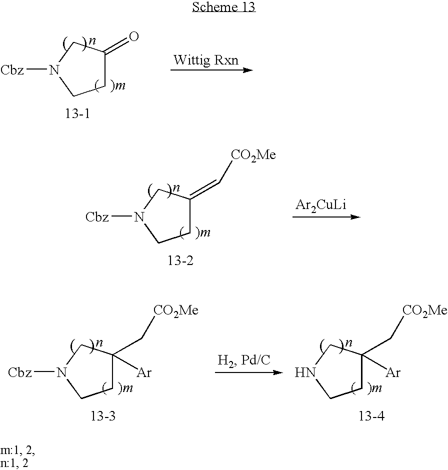

Pyrrolidine 14-4 can be prepared according to Scheme 14. Halogen metal exchange between aryl iodide 14-1 and isopropylmagnesium bromide followed by reaction with N-Boc-3-oxo-pyrrolidine 14-2 provides spiral lactone 14-3, which upon acidic cleavage of the Boc group yields the desired pyrrolidine 14-4.

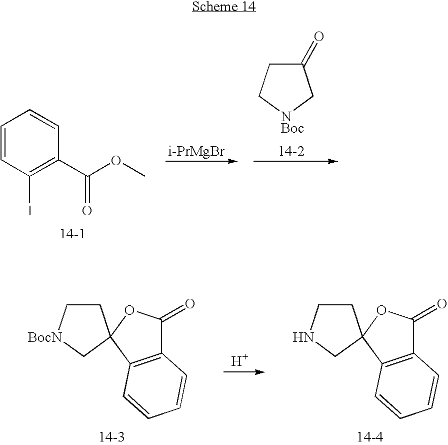

Pyrrolidine 15-4 can be prepared according to the method outlined in Scheme 15. Ortho lithiation of carboxylic acid 15-1 with n-butyl lithium (n-BuLi) or lithium 2,2,6,6-tetramethylpiperidide (LTMP), followed by reaction of the resulting organolithium with N-Boc-3-oxo-pyrrolidine 15-2 yields spiral lactone 15-3, which upon acidic cleavage of the Boc group provides the desired pyrrolidine 15-4.

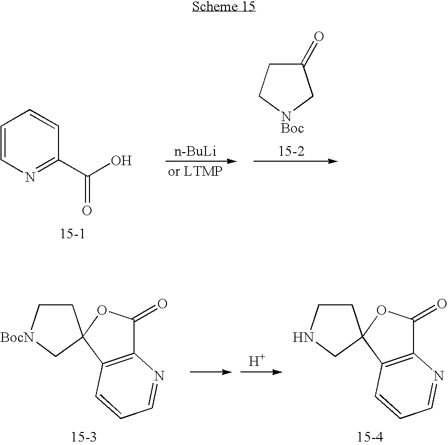

A series of compounds 16-5 can be prepared by the method outlined in Scheme 16. Compound 16-1 can be alkylated (with R²Br or R²I; wherein R² is alkyl, arylalkyl, cycloalkyl or the like) in the standard fashion as has been described previously to give the desired alkylated product 16-2. Both benzyl groups (Bn) of 43 can be removed by hydrogenation to give the deprotected compound 16-3. Treatment of the compound 16-3 with a primary or secondary amine HNR³R⁴ can provide an amide 16-4. The free hydroxyl group of 16-4 can be converted to a variety of ether analogs 16-5 by routine methods wherein R can be alkyl, aryl, cycloalkyl, arylalkyl or other suitable groups.

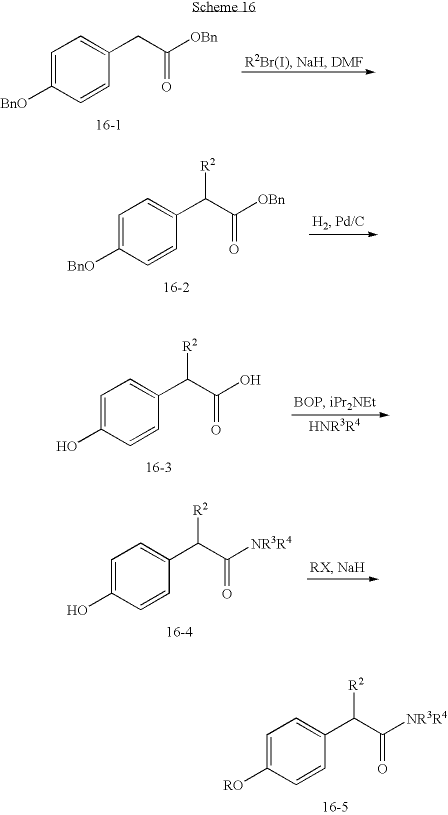

A series of compounds 17-3 (wherein Ar is an aryl or heteroaryl group which is optionally substituted by one or more substitutents such as halo, alkyl, etc.) can be prepared by the method outlined in Scheme 17. A phenol 17-1 can be converted to the corresponding the triflate 17-2 which then can undergo Pd catalyzed Suzuki coupling with a boronic acid ArB(OH)₂ or a derivative thereof to provide a compound 17-3.

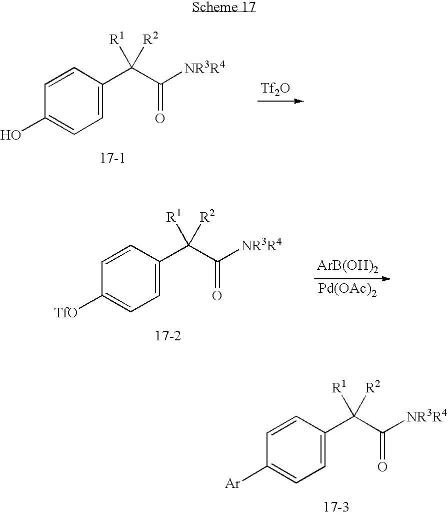

A series of compounds 18-2 (wherein Ar is an aryl or heteroaryl group which is optionally substituted by one or more substitutents such as halo, alkyl, etc.) can be prepared by the method outlined in Scheme 18. The free OH group of the phenol 18-1 can be coupled with a boronic acid ArB(OH)₂ or a derivative thereof directly to provide the aryl or heteroaryl ether coupling product 18-2.

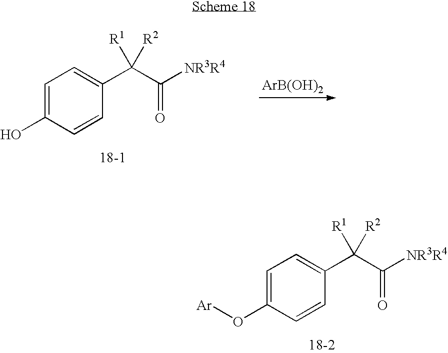

A series of heterocycloalkyl- or heterocylcoalkylalkyl-ether compounds 19-4 and 19-5 can be prepared by the method outlined in Scheme 19. The free phenol of 19-1 can be treated with a variety of heterocycloalkyl triflates 19-2 or heterocycloalkylalkyl halides 19-3 to provide the heterocycloalkyl- or heterocylcoalkylalkyl-ether compounds 19-4 and 19-5 respectively. 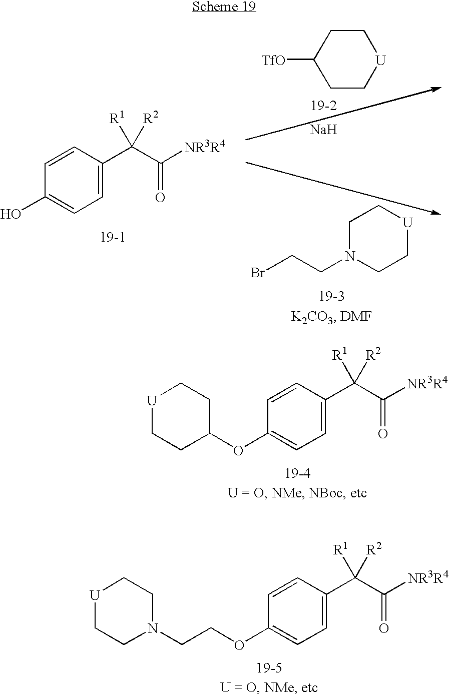
Methods

Compounds of the invention can modulate activity of 11βHSD1. The term “modulate” is meant to refer to an ability to increase or decrease activity of an enzyme. Accordingly, compounds of the invention can be used in methods of modulating 11βHSD1 by contacting the enzyme with any one or more of the compounds or compositions described herein. In some embodiments, compounds of the present invention can act as inhibitors of 11βHSD1. In further embodiments, the compounds of the invention can be used to modulate activity of 11βHSD1 in an individual in need of modulation of the enzyme by administering a modulating amount of a compound of the invention.

The present invention further provides methods of inhibiting the conversion of cortisone to cortisol in a cell, or inhibiting the production of cortisol in a cell, where conversion to or production of cortisol is mediated, at least in part, by 11βHSD1 activity. Methods of measuring conversion rates of cortisone to cortisol and vice versa, as well as methods for measuring levels of cortisone and cortisol in cells, are routine in the art.

The present invention further provides methods of increasing insulin sensitivity of a cell by contacting the cell with a compound of the invention. Methods of measuring insulin sensitivity are routine in the art.

The present invention further provides methods of treating disease associated with activity or expression, including abnormal activity and overexpression, of 11βHSD1 in an individual (e.g., patient) by administering to the individual in need of such treatment a therapeutically effective amount or dose of a compound of the present invention or a pharmaceutical composition thereof. Example diseases can include any disease, disorder or condition that is directly or indirectly linked to expression or activity of the enzyme. An 11βHSD1-associated disease can also include any disease, disorder or condition that can be prevented, ameliorated, or cured by modulating enzyme activity.

Examples of 11βHSD1-associated diseases include obesity, diabetes, glucose intolerance, insulin resistance, hyperglycemia, atherosclerosis, hypertension, hyperlipidemia, cognitive impairment, dementia, depression (e.g., psychotic depression), glaucoma, cardiovascular disorders, osteoporosis, and inflammation. Further examples of 11βHSD1-associated diseases include metabolic syndrome, coronary heart disease, type 2 diabetes, hypercortisolemia, androgen excess (hirsutism, menstrual irregularity, hyperandrogenism) and polycystic ovary syndrome (PCOS).

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal. In some embodiments, the cell is an adipocyte, a pancreatic cell, a hepatocyte, neuron, or cell comprising the eye.

As used herein, the term “contacting” refers to the bringing together of indicated moieties in an in vitro system or an in vivo system. For example, “contacting” the 11βHSD1 enzyme with a compound of the invention includes the administration of a compound of the present invention to an individual or patient, such as a human, having 11βHSD1, as well as, for example, introducing a compound of the invention into a sample containing a cellular or purified preparation containing the 11βHSD1 enzyme.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder; and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

Pharmaceutical Formulations and Dosage Forms

When employed as pharmaceuticals, the compounds of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral adminstration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, antibodies, immune suppressants, anti-inflammatory agents and the like.

Labeled Compounds and Assay Methods

Another aspect of the present invention relates to labeled compounds of the invention (radio-labeled, fluorescent-labeled, etc.) that would be useful not only in radio-imaging but also in assays, both in vitro and in vivo, for localizing and quantitating the enzyme in tissue samples, including human, and for identifying ligands by inhibition binding of a labeled compound. Accordingly, the present invention includes enzyme assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), 3H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, 123I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, 14C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from ³H, ¹⁴C, ¹²⁵I , ³⁵S and ⁸²Br.

In some embodiments, the labeled compounds of the present invention contain a fluorescent lable.

Synthetic methods for incorporating radio-isotopes and fluorescent labels into organic compounds are are well known in the art.

A labeled compound of the invention (radio-labeled, fluorescent-labeled, etc.) can be used in a screening assay to identify/evaluate compounds. For example, a newly synthesized or identified compound (i.e., test compound) which is labeled can be evaluated for its ability to bind a 11βHSD1 by monitering its concentration variation when contacting with the 11βHSD1, through tracking the labeling. For another example, a test compound (labeled) can be evaluated for its ability to reduce binding of another compound which is known to bind to 11βHSD1 (i.e., standard compound). Accordingly, the ability of a test compound to compete with the standard compound for binding to the 11βHSD1 directly correlates to its binding affinity. Conversely, in some other screening assays, the standard compound is labled and test compounds are unlabeled. Accordingly, the concentration of the labled standard compound is monitored in order to evaluate the competition between the standard compound and the test compound, and the relative binding affinity of the test compound is thus ascertained.

Kits

The present invention also includes pharmaceutical kits useful, for example, in the treatment or prevention of 11βHSD1-associated diseases or disorders, obesity, diabetes and other diseases referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. Certain compounds of the Examples were found to be inhibitors of 11βHSD1 according to one or more of the assays provided herein.

### EXAMPLES

### Example 1

### 1′-[(4-Bromo-2-fluorophenyl)(hydroxy)acetyl]-3H-spiro[2-benzofuran-1,3′-pyrrolidin]-3-one

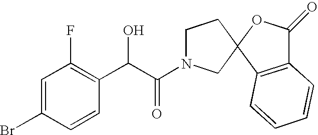

To a mixture of (7,7-dimethyl-2-oxobicyclo[2.2.1]hept-1-yl)methanesulfonic acid-3H-spiro[2-benzofuran-1,3′-pyrrolidin]-3-one (1:1) (1.269 g, 0.003011 mol), (4-bromo-2-fluorophenyl)(hydroxy)acetic acid (0.750 g, 0.00301 mol) in N,N-dimethylformamide (9.648 mL) was added benzotriazol-1-yloxytris(dimethylamino)phosphonium hexafluorophosphate (1.598 g, 0.003613 mol). After stirring at rt for 10 min, the mixture was treated with N,N-diisopropylethylamine (1.311 mL, 0.007528 mol) at 0 ° C and then stirred at rt for 2 h. The mixture was diluted with water, and extracted with EtOAc. The organic layers were combined, washed with 1 N NaOH and brine successively, dried and evaporated to dryness. The residue was purified on silica gel, eluting with 0 to 80% EtOAc in hexane, to give the product (1.08 g, 85.34%). LCMS (M+H) 420.0.

### Example 2

### 1′-[(4-Bromo-2-fluorophenyl)(methoxy)acetyl]-3H-spiro[2-benzofuran-1,3′-pyrrolidin]-3-one

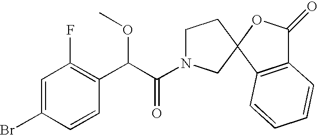

To a mixture of 1′-[(4-bromo-2-fluorophenyl)(hydroxy)acetyl]-3H-spiro[2-benzofuran-1,3′-pyrrolidin]-3-one (0.85 g, 0.0020 mol) in N,N-dimethylformamide (8.00 mL) was added sodium hydride (0.101 g, 0.00253 mol). After stirring at rt for 20 min, to the resultant mixture was added methyl iodide (0.189 mL, 0.00303 mol). The reaction mixture was stirred at rt for 3 h, then quenched with aq. ammonium chloride. The mixture was extracted with EtOAc. The combined organic layers were washed with brine, dried and evaporated to dryness. The residue was purified on silica gel, eluting with 0 to 50% EtOAc in hexane, to afford the methyl ether (800 mg, 91.08%). LCMS (M+H) 434.0.

### Example 3

### 5-(3-Fluoro-4-1-methoxy-2-oxo-2-[3-oxo-1′H,3H-spiro[2-benzofuran-1,3′-pyrrolidin]-1′-yl]ethylphenyl)-N-methylpyridine-2-carboxamide

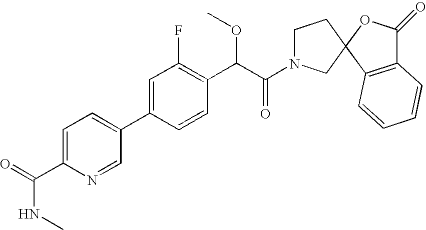

A mixture of 1′-[(4-bromo-2-fluorophenyl)(methoxy)acetyl]-3H-spiro[2-benzofuran-1,3′-pyrrolidin]-3-one (20.0 mg, 0.0000460 mol), N-methyl-5-(4,4,5,5-tetramethyl-1,3,2-dioxaborolan-2-yl)pyridine-2-carboxamide (18.1 mg, 0.0000691 mol) and potassium carbonate (19.1 mg, 0.000138 mol) in N,N-dimethylformamide (0.39 mL) was purged with nitrogen for 5 min. After an addition of [1,1′-bis(diphenylphosphino)ferrocene]dichloropalladium(II),complex with dichloromethane (1:1) (5.64 mg, 6.91E-6 mol), the resulting mixture was heated at 100° C. for 4 h. The reaction mixture was diluted with AcCN and water, filtered through a 0.3 U membrane. The filtrate was applied on RP-HPLC to yield the desired product (15 mg, 66.54%). The product was believed to be in the form of a trifluoroacetic acid salt. LCMS (M+H) 490.1.

### Example 4

### 1′-[2-(4-bromo-2-fluorophenyl)-2-methoxybutanoyl]-3H-spiro[2-benzofuran-1,3′-pyrrolidin]-3-one

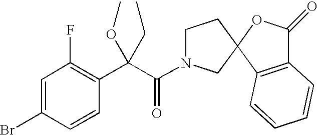

### Step 1. 1′-[(4-bromo-2-fluorophenyl)(oxo)acetyl]-3H-spiro[2-benzofuran-1,3′-pyrrolidin]-3-one

Dimethyl sulfoxide (0.558 mL, 0.00786 mol) was added to a solution of oxalyl chloride (0.332 mL, 0.00393 mol) in methylene chloride (20.0 mL, 0.312 mol) at −78° C. After 10 min, a solution of 1′-[(4-bromo-2-fluorophenyl)(hydroxy)acetyl]-3H-spiro[2-benzofuran-1,3′-pyrrolidin]-3-one (1.38 g, 0.00327 mol) in methylene chloride (10.0 mL, 0.156 mol) was added and the resultant mixture was stirred at −78° C. for 30 min. Triethylamine (2.28 mL, 0.0164 mol) was then added and the mixture was stirred for 5 h with the reaction temperature allowed to gradually warm up to rt. After quenched with water, the mixture was extracted with methylene chloride. The organic layers were combined, washed with brine, dried and evaporated to dryness. The residue was crystallized from methylene chloride to give the pure keton compound. The mother liquor was concentrated to dryness and purified on silica gel, eluting with 0 to 60% EtOAc in hexane to yield additional product (total: 1.18 g, 86.16%). LCMS (M+H) 418.0.

### Step 2. 1′-[2-(4-bromo-2-fluorophenyl)-2-hydroxybutanoyl]-3H-spiro[2-benzofuran-1,3′-pyrrolidin]-3-one

To a suspension of 1′-[(4-bromo-2-fluorophenyl)(oxo)acetyl]-3H-spiro[2-benzofuran-1,3′-pyrrolidin]-3-one (1.00 g, 0.00239 mol) in tetrahydrofuran (40.00 mL, 0.4932 mol) was added a solution of ethylmagnesium bromide in ether (3.00 M, 1.00 ML) dropwise at 0° C. The reaction mixture was stirred at rt for 2 h, quenched with aq. ammonium chloride, and then extracted with EtOAc. The combined organic layers were washed with brine, dried, and evaporated to dryness. The residue was purified on silica gel, eluting with 0 to 60% EtOAc in hexane, to give the desired product together with some hydrated starting material. The mixture was used directly in next step. LCMS (M+H) 448.0.

### Step 3. 1′-[2-(4-bromo-2-fluorophenyl)-2-methoxybutanoyl]-3H-spiro[2-benzofuran-1,3′-pyrrolidin]-3-one

To a mixture of 1′-[2-(4-bromo-2-fluorophenyl)-2-hydroxybutanoyl]-3H-spiro[2-benzofuran-1,3′-pyrrolidin]-3-one (20.0 mg, 0.0000446 mol) in N,N-dimethylformamide (0.50 mL) was added sodium hydride (2.68 mg, 0.0000669 mol). After stirring at rt for 30 min, to the mixture was added methyl iodide (0.00347 mL, 0.0000558 mol). The resultant mixture was stirred at rt for an additional 3 h, then quenched with 1 N HCl. The mixture was purified on RP-HPLC to yield the desired compound (9 mg, 43.63%). LCMS (M+H) 462.0.

### Example 5

### 2-(4-Bromo-2-fluorophenyl)-2-hydroxy-N-methyl-N-(tetrahydro-2H-pyran-4-yl)acetamide

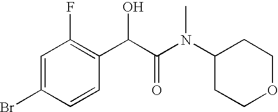

To a mixture of N-methyltetrahydro-2H-pyran-4-amine hydrochloride (0.3044 g, 0.002007 mol) and (4-bromo-2-fluorophenyl)(hydroxy)acetic acid (0.500 g, 0.00201 mol) in N,N-dimethylformamide (6.432 mL) was added benzotriazol-1-yloxytris(dimethylamino)phosphonium hexafluorophosphate (1.065 g, 0.002409 mol). After stirring at rt for 10 min, the mixture was treated with N,N-diisopropylethylamine (0.8742 mL, 0.005019 mol) at 0° C. and then stirred at rt for 2 h. The mixture was diluted with water, and then extracted with EtOAc. The organic layers were combined, washed with 1 N NaOH and brine successively, dried and evaporated to dryness. The residue was purified on silica gel, eluting with 0 to 80% EtOAc in hexane, to give the product (568 mg, 81.73%). LCMS (M+H) 346.0.

### Example 6

### 1-(4-Bromo-2-fluorophenyl)-2-oxo-2-(1,3,3-trimethyl-6-azabicyclo[3.2.1]oct-6-yl)ethanol

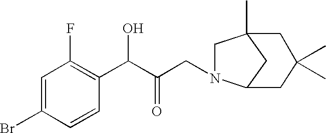

To a mixture of 1,3,3-trimethyl-6-azabicyclo[3.2.1]octane (0.3077 g, 0.002008 mol), (4-bromo-2-fluorophenyl)(hydroxy)acetic acid (0.500 g, 0.00201 mol) in N,N-dimethylformamide (6.432 mL) was added benzotriazol-1-yloxytris(dimethylamino)phosphonium hexafluorophosphate (1.065 g, 0.002409 mol). After stirring at rt for 10 min, the mixture was treated with N,N-diisopropylethylamine (0.667 mL, 0.00383 mol) at 0° C. and then stirred at rt for 2 h. The mixture was diluted with water and then extracted with EtOAc. The organic layers were combined, washed with 1 N NaOH and brine successively, dried and evaporated to dryness. The residue was purified on silica gel, eluting with 0 to 40% EtOAc in hexane, to give the product (684 mg, 88.65%). LCMS (M+H) 384.1.

### Example 7

### 6-[(4-Bromo-2-fluorophenyl)(methoxy)acetyl]-1,3,3-trimethyl-6-azabicyclo [3.2.1]octane

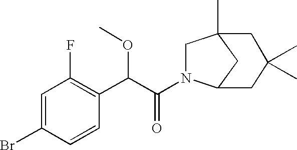

To a mixture of 1-(4-bromo-2-fluorophenyl)-2-oxo-2-(1,3,3-trimethyl-6-azabicyclo[3.2.1]oct-6-yl)ethanol (24.0 mg, 0.0000624 mol) in N,N-dimethylformamide (0.300 mL, 0.00388 mol) was added sodium hydride (3.75 mg, 0.0000937 mol). After stirred at rt for 30 min, to the mixture was added methyl iodide (0.00486 mL, 0.0000781 mol). The reaction mixture was stirred for an additional 2 h, then neutralized with 1N HCl and purified on RP-HPLC to yield the desired product (20 mg, 80.4%).-LCMS (M+H) 398.1.

### Example 8

### 2-(4-Bromophenyl)-N-(4-hydroxycyclohexyl)-N-methylpropanamide

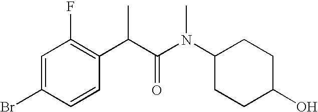

### Step 1. ethyl 2-(4-bromophenyl)propanoate

To a mixture of ethyl (4-bromophenyl)acetate (10.00 g, 0.04114 mol) in N,N-dimethylformamide (100.00 mL) was added sodium hydride (2.468 g, 0.06170 mol) at 0° C. The resulting orange-red mixture was stirred at 0° C. for 30 min. To the mixture was then added methyl iodide (3.201 mL, 0.05142 mol). The orange-red color of the mixture faded upon the completion of the addition of methyl iodide. The reaction mixture was stirred at rt for 1 h, quenched with aq. ammonium and then extracted with EtOAc. The combined organic layers were washed with water and brine successively, dried, and evaporated to dryness. The residue was purified by column chromatography on silica gel, eluting with 0 to 20% EtOAc, to give the desired product (4.88 g, 46.14%). LCMS (M+H) 257.0.

### Step 2. 2-(4-bromophenyl)propanoic Acid

To a mixture of ethyl 2-(4-bromophenyl)propanoate (5.00 g, 0.0194 mol) in tetrahydrofuran (100.0 mL) was added a solution of lithium hydroxide (2.33 g, 0.0972 mol) in water (50.0 mL). The mixture was stirred at rt for 2 h. After stripping off THF under reduced pressure, the remaining aqueous layer was acidified with 6 N HCl at 0° C. The resultant mixture was extracted with EtOAc. The organic layers were combined, washed with water and brine successively, dried over magnesium sulfate, and evaporated to dryness. The crude white solid was used directly in next step (4.30 g, 96.53%). LCMS (M+H) 229.0.

### Step 3. cis-4-(methylamino)cyclohexanol Hydrochloride

To a suspension of lithium tetrahydroaluminate (2.70 g, 0.0711 mol) in tetrahydrofuran (120.0 mL, 1.479 mol) was added tert-butyl (cis-4-hydroxycyclohexyl)carbamate (3.00 g, 0.0139 mol). The reaction mixture was heated at reflux overnight. After cooling to rt, the mixture was carefully quenched with additions of water (2.70 mL, 0.150 mol), a solution of sodium hydroxide in water (3.75 M, 2.70 mL) (15%) and water (8.100 mL, 0.4496 mol) successively. After stirring at rt for 1 h, the mixture was filtered through a pad of Celite. The filtrate was dried over magnesium sulfate and evaporated to dryness. LCMS (M+H) 130.2. The crude amine was treated with 40 mL of 4 M HCl in dioxane solution at rt for 4 h, then evaporated to dryness to afford the corresponding HCl salt (2.16 g, 93.57%).

### Step 4. 2-(4-bromophenyl)-N-(4-hydroxycyclohexyl)-N-methylpropanamide

To a mixture of 2-(4-bromophenyl)propanoic acid (30.0 mg, 0.000131 mol) and benzotriazol-1-yloxytris(dimethylamino)phosphonium hexafluorophosphate (72.4 mg, 0.000164 mol) in N,N-dimethylformamide (0.500 mL) was added cis-4-(methylamino)cyclohexanol hydrochloride, followed by N,N-diisopropylethylamine (0.0684 mL, 0.000393 mol). The reaction mixture was stirred at rt for 1 h. The resultant mixture was purified on RP-HPLC to yield the desired amide believed to have a cis configuration (38 mg, 85.28%). LCMS (M+H) 340.1.

### Example 9

### 8-[2-(4-Bromophenyl)propanoyl]-8-azabicyclo[3.2.1]octan-3-ol

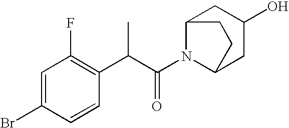

To a mixture of 2-(4-bromophenyl)propanoic acid (30.0 mg, 0.000131 mol) and benzotriazol-1-yloxytris(dimethylamino)phosphonium hexafluorophosphate (72.4 mg, 0.000164 mol) in N,N-dimethylformamide (0.500 mL, 0.00646 mol) was added (3-endo)-8-azabicyclo[3.2.1]octan-3-ol hydrochloride, followed by N,N-diisopropylethylamine (0.0684 mL, 0.000393 mol). The reaction mixture was stirred at rt for 1 h. The resultant mixture was purified on RP-HPLC to yield the desired amide which was believed to have an endo configuration (38 mg, 85.78%). LCMS (M+H) 338.1.

### Example 10

### 1′-12-(4-Bromophenyl)propanoyl]-3H-spiro[2-benzofuran-1,3′-pyrrolidin]-3-one

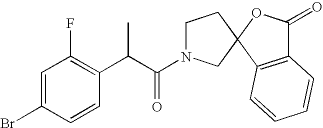

To a mixture of 2-(4-bromophenyl)propanoic acid (30.0 mg, 0.000131 mol), and benzotriazol-1-yloxytris(dimethylamino)phosphonium hexafluorophosphate (72.4 mg, 0.000164 mol) in N,N-dimethylformamide (0.500 mL, 0.00646 mol) was added (7,7-dimethyl-2-oxobicyclo[2.2.1]hept-1-yl)methanesulfonic acid-3H-spiro[2-benzofuran-1,3′-pyrrolidin]-3-one (1:1), followed by N,N-diisopropylethylamine (0.0684 mL, 0.000393 mol). The reaction mixture was stirred at rt for 1 h. The resultant mixture was purified on RP-HPLC to yield the desired amide (44 mg, 83.94%). LCMS (M+H) 400.0.

### Example 11

### N-Methyl-5-(4-1-methyl-2-oxo-2-[3-oxo-1′H,3H-spiro[2-benzofuran-1,3′-pyrrolidin]-1′-yl]ethylphenyl)pyridine-2-carboxamide

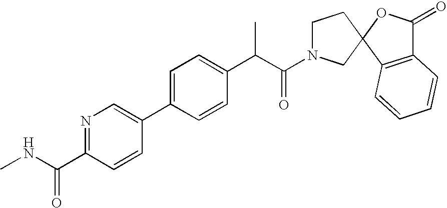

A mixture of 1′-[2-(4-bromophenyl)propanoyl]-3H-spiro[2-benzofuran-1,3′-pyrrolidin]-3-one (30.0 mg, 0.0000750 mol), N-methyl-5-(4,4,5,5-tetramethyl-1,3,2-dioxaborolan-2-yl)pyridine-2-carboxamide (29.5 mg, 0.000112 mol) and potassium carbonate (31.1 mg, 0.000225 mol) in N,N-dimethylformamide (0.600 mL, 0.00775 mol) was purged with nitrogen for 5 min. After an addition of [1,1′-bis(diphenylphosphino)ferrocene]dichloropalladium(II), complex with dichloromethane (1:1) (9.18 mg, 0.0000112 mol), the resulting mixture was heated at 120° C. for 4 h. The reaction mixture was diluted with AcCN and water, filtered through a 0.3 U membrane. The filtrate was applied on RP-HPLC to generate the desired product (23 mg, 67.37%). The product was believed to be in the form of a trifluoroacetic acid salt. LCMS (M+H) 456.2.

### Example 12

### 5-(4-(2-[3-Hydroxy-8-azabicyclo[3.2.1]oct-8-yl]-1-methyl-2-oxoethyl)phenyl)-N-methylpyridine-2-carboxamide

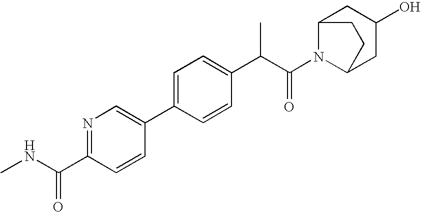

This compound was prepared by using a procedure analogous to that described for the synthesis of example 11 and was believed to have an endo configuration. The product was believed to be in the form of a trifluoroacetic acid salt. LCMS (M+H) 394.2.

### Example 13

### 5-(4-(2-[(4-Hydroxycyclohexyl)(methyl)amino]-1-methyl-2-oxoethyl)phenyl)-N-methylpyridine-2-carboxamide

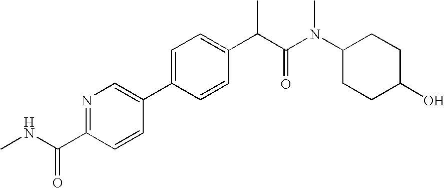

This compound was prepared by using procedures analogous to those described for the synthesis of example 11, and the compound was believed to have a cis configuration. LCMS (M+H) 396.2.

### Example 14

### 2-(4-Bromo-2-fluorophenyl)-2-fluoro-N-methyl-N-(tetrahydro-2H1-pyran-4-yl)acetamide

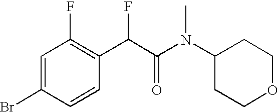

To a mixture of 2-(4-bromo-2-fluorophenyl)-2-hydroxy-N-methyl-N-(tetrahydro-2H-pyran-4-yl)acetamide (20.0 mg, 0.0000578 mol) in dichloromethane (0.514 mL, 0.00801 mol) at 0° C. was added 2-methoxy-N-(2-methoxyethyl)-N-(trifluoro-λ(4)-sulfanyl)ethanamine (0.0298 g, 0.000135 mol) dropwise. The resultant mixture was allowed to warm up to rt and stirred at 40° C. overnight. After cooling to rt, the mixture was poured into aq. sodium bicarbonate, and then extracted with methylene chloride. The combined organic layers were dried (over sodium sulfate), and evaporated to dryness. The residue was purified on RP-HPLC to give the desired product (17 mg, 84.51%). The product was believed to be in the form of a trifluoroacetic acid salt. LCMS (M+H) 348.1.

### Example 15

### 6-[(4-Bromo-2-fluorophenyl)(fluoro)acetyl]-1,3,3-trimethyl-6-azabicyclo[3.2.1]octane

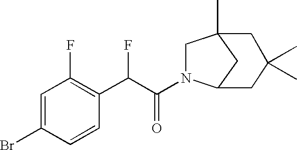

This compound was prepared by using a procedure analogous to that described for the synthesis of example 14. LCMS (M+H) 386.1.

### Example 16

### 6-[(4-Bromo-2-fluorophenyl)(difluoro)acetyl]-1,3,3-trimethyl-6-azabicyclo[3.2.1]octane

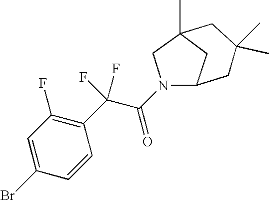

### Step 1. 1-(4-bromo-2-fluorophenyl)-2-oxo-2-(1,3,3-trimethyl-6-azabicyclo[3.2.1]oct-6-yl)ethanone

Dimethyl sulfoxide (0.443 mL, 0.00624 mol) was added to oxalyl chloride (0.264 mL, 0.00312 mol) in methylene chloride (15.9 mL, 0.248 mol) at −78° C. After 10 min, 1-(4-bromo-2-fluorophenyl)-2-oxo-2-(1,3,3-trimethyl-6-azabicyclo[3.2.1]oct-6-yl)ethanol (1.00 g, 0.00260 mol) in methylene chloride (7.95 mL, 0.124 mol) was added and the resultant mixture was stirred at −78° C. for 30 min. Triethylamine (1.81 mL, 0.0130 mol) was then added and the mixture was stirred for 5 h during which the temperature allowed to gradually warm up to room temperature (rt). After quenching with water, the mixture was extracted with methylene chloride. The organic layers were combined, washed with brine, dried and evaporated to dryness. The residue was crystalized from methylene chloride to give pure ketone product. The mother liquor was concentrated to dryness and purified on silica gel, eluting with 0 to 60% EtOAc in hexane to yield additional product. Total yield: 896 mg (90.07%). LCMS (M+H) 382.1.

### Step 2. 6-[(4-bromo-2-fluorophenyl)(difluoro)acetyl]-1,3,3-trimethyl-6-azabicyclo[3.2.1]octane

To a mixture of 1-(4-bromo-2-fluorophenyl)-2-oxo-2-(1,3,3-trimethyl-6-azabicyclo[3.2.1]oct-6-yl)ethanone (10.0 mg, 0.0000262 mol) in methylene chloride (0.143 mL, 0.00223 mol) at 0° C. was added 2-methoxy-N-(2-methoxyethyl)-N-(trifluoro-λ(4)-sulfanyl)ethanamine (0.0143 mL, 0.0000775 mol) dropwise. The resultant mixture was allowed to warm up to rt and stirred at 40° C. overnight. An additional 0.1 mL of 2-methoxy-N-(2-methoxyethyl)-N-(trifluoro-λ(4)-sulfanyl)ethanamine was added. The mixture was stirred at rt for another 3 d. The mixture was poured into aq. sodium bicarbonate and extracted with methylene chloride. The combined organic layers were dried (sodium sulfate) the evaporated to dryness. The residue was purified on RP-HPLC to give the desired product (5 mg, 47.28%). LCMS (M+H) 404.1.

### Example 17

### 2-(4-Bromo-2-fluorophenyl)-1-oxo-1-(1,3,3-trimethyl-6-azabicyclo[3.2.1]oct-6yl)propan-2-ol

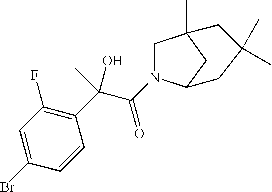

To a mixture of 1-(4-bromo-2-fluorophenyl)-2-oxo-2-(1,3,3-trimethyl-6-azabicyclo[3.2.1]oct-6-yl)ethanone (0.817 g, 0.00214 mol) in tetrahydrofuran (10.47 mL, 0.1291 mol) at 0° C. was added 1.400 M of methylmagnesium bromide in tetrahydrofuran (1.908 mL) dropwise. The resultant mixture was allowed to warm up to rt and stirred at 40° C. overnight. After cooling to rt, the mixture was poured into aq. sodium bicarbonate and then extracted with methylene chloride. The combined organic layers were dried (sodium sulfate) and then evaporated to dryness. The residue was purified on silica gel, eluting with 0 to 40% EtOAc in hexane, to give the desired product (782 mg, 91.86%). LCMS (M+H) 398.1.

### Example 18

### 5-(3-fluoro-4-[1-fluoro-2-oxo-2-(1,3,3-trimethyl-6-azabicyclo[3.2.1]oct-6-yl)ethyl]phenyl)-N-methylpyridine-2-carboxamide

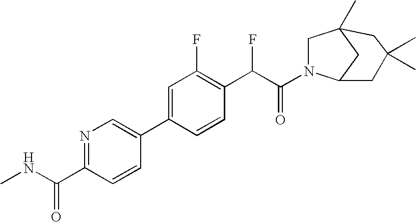

A mixture of 6-[(4-bromo-2-fluorophenyl)(fluoro)acetyl]-1,3,3-trimethyl-6-azabicyclo[3.2.1]octane (25.0 mg, 0.0000647 mol), N-methyl-5-(4,4,5,5-tetramethyl-1,3,2-dioxaborolan-2-yl)pyridine-2-carboxamide (25.4 mg, 0.0000971 mol) and potassium carbonate (26.8 mg, 0.000194 mol) in N,N-dimethylformamide (0.518 mL, 0.00669 mol) was degassed with nitrogen for 5 min. To the mixture was added [1,1′-bis(diphenylphosphino)ferrocene]dichloropalladium(II) complexed with dichloromethane (1:1) (7.93 mg, 9.71E-6 mol). The resulting mixture was then heated at 120° C. for 4 h. The reaction mixture was diluted with acetonitrile and water and then filtered through a 0.3 U membrane. The filtration was applied on RP-HPLC to generate the desired product (18 mg, 62.99%). The product was believed to be in the form of a trifluoroacetic acid salt. LCMS (M+H) 442.2.

### Example 19

### 5-(3-Fluoro-4-[1-hydroxy-1-methyl-2-oxo-2-(1,3,3-trimethyl-6-azabicyclo[3.2.1]oct-6-yl)ethyl]phenyl)-N-methylpyridine-2-carboxamide

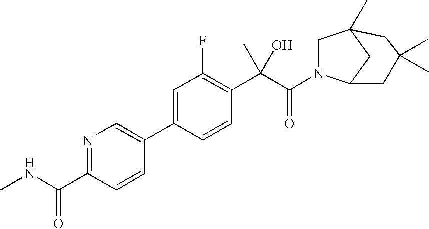

A mixture of 2-(4-bromo-2-fluorophenyl)-1-oxo-1-(1,3,3-trimethyl-6-azabicyclo[3.2.1]oct-6-yl)propan-2-ol (25.0 mg, 0.0000628 mol), N-methyl-5-(4,4,5,5-tetramethyl-1,3,2-dioxaborolan-2-yl)pyridine-2-carboxamide (24.7 mg, 0.0000941 mol) and potassium carbonate (26.0 mg, 0.000188 mol) in N,N-dimethylformamide (0.502 mL, 0.00649 mol) was degassed with nitrogen for 5 min. To the mixture was added [1,1′-bis(diphenylphosphino)ferrocene]dichloropalladium(II) complexed with dichloromethane (1:1) (7.69 mg, 9.41E-6 mol). The resulting mixture was heated at 120° C. for 4 h. The reaction mixture was diluted with AcCN and water and filtered through a 0.3 U membrane. The filtration was applied on RP-HPLC to generate the desired product (21 mg, 73.77%). The product was believed to be in the form of a trifluoroacetic acid salt. LCMS (M+H) 454.2.

### Example 20

### 6-[2-(4-Bromo-2-fluorophenyl)-2-methoxypropanoyl]-1,3,3-trimethyl-6-azabicyclo[3.2.1]octane

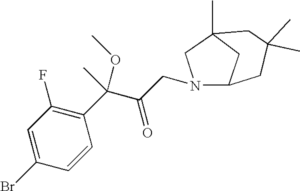

To a mixture of 2-(4-bromo-2-fluorophenyl)-1-oxo-1-(1,3,3-trimethyl-6-azabicyclo-[3.2.1]oct-6-yl)-propan-2-ol (150.0 mg, 0.0003766 mol) in N,N-dimethylformamide (0.857 mL, 0.0111 mol) was added sodium hydride (22.59 mg, 0.0005649 mol). The resulting mixture was stirred at rt for 30 min, then treated with methyl iodide (0.03517 mL, 0.0005649 mol) at rt for an additional 3 h. The reaction mixture was diluted with AcCN and water and then filtered through a 0.3 U membrane. The filtration was applied on RP-HPLC to generate the desired product (139 mg, 89.51%). LCMS (M+H) 412.1.

### Example 21

### 6-[2-(4-Bromo-2-fluorophenyl)-2-fluoropropanoyl]-1,3,3-trimethyl-6-azabicyclo[3.2.1]octane

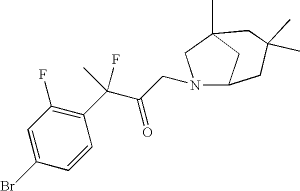

To a mixture of 2-(4-bromo-2-fluorophenyl)-1-oxo-1-(1,3,3-trimethyl-6-azabicyclo[3.2.1]oct-6-yl)propan-2-ol (50.0 mg, 0.000126 mol) in methylene chloride (0.750 mL, 0.0117 mol) at 0° C. was added 2-methoxy-N-(2-methoxyethyl)-N-(trifluoro-λ(4)-sulfanyl)ethanamine (0.0539 mL, 0.000292 mol) dropwise. The resultant mixture was allowed to warm up to rt and stirred at 40° C. overnight. After cooling to rt, the mixture was poured into aq. sodium bicarbonate and extracted with methylene chloride. The combined organic layers were dried (sodium sulfate) and then evaporated to dryness. The residue was purified on RP-HPLC to give the desired product (27 mg, 53.73%). LCMS (M+H) 400.0.

### Example A

### Enzymatic Assay of 11βHSD1

All in vitro assays were performed with clarified lysates as the source of 11βHSD1 activity. HEK-293 transient transfectants expressing an epitope-tagged version of full-length human 11βHSD1 were harvested by centrifugation. Roughly 2×10⁷ cells were resuspended in 40 mL of lysis buffer (25 mM Tris-HCl, pH 7.5, 0.1 M NaCl, 1 mM MgCl₂ and 250 mM sucrose) and lysed in a microfluidizer. Lysates were clarified by centrifugation and the supernatants were aliquoted and frozen.

Inhibition of 11βHSD1 by test compounds was assessed in vitro by a Scintillation Proximity Assay (SPA). Dry test compounds were dissolved at 5 mM in DMSO. These were diluted in DMSO to suitable concentrations for the SPA assay. 0.8 μL of 2-fold serial dilutions of compounds were dotted on 384 well plates in DMSO such that 3 logs of compound concentration were covered. 20 μL of clarified lysate was added to each well. Reactions were initiated by addition of 20 μL of substrate-cofactor mix in assay buffer (25 mM Tris-HCl, pH 7.5, 0.1 M NaCl, 1 mM MgCl₂) to final concentrations of 400 μM NADPH, 25 nM ³H-cortisone and 0.007% Triton X-100. Plates were incubated at 37° C. for one hour. Reactions were quenched by addition of 40 μL of anti-mouse coated SPA beads that had been pre-incubated with 10 μM carbenoxolone and a cortisol-specific monoclonal antibody. Quenched plates were incubated for a minimum of 30 minutes at RT prior to reading on a Topcount scintillation counter. Controls with no lysate, inhibited lysate, and with no mAb were run routinely. Roughly 30% of input cortisone is reduced by 11βHSD1 in the uninhibited reaction under these conditions.

Test compounds having an IC₅₀ value less than about 100 μM according to this assay were considered active. The compound of Example 1 was found to have an IC₅₀ value of less than 1 μM.

### Example B

### Cell-Based Assays for HSD Activity

Peripheral blood mononuclear cells (PBMCs) were isolated from normal human volunteers by Ficoll density centrifugation. Cells were plated at 4×10⁵ cells/well in 200 μL of AIM V (Gibco-BRL) media in 96 well plates. The cells were stimulated overnight with 50 ng/ml recombinant human IL-4 (R&D Systems). The following morning, 200 nM cortisone (Sigma) was added in the presence or absence of various concentrations of compound. The cells were incubated for 48 hours and then supernatants were harvested. Conversion of cortisone to cortisol was determined by a commercially available ELISA (Assay Design).

Test compounds having an IC₅₀ value less than about 100 μM according to this assay were considered active.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula I or Ia: 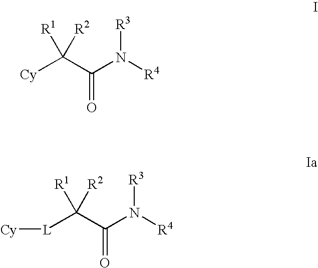
or pharmaceutically acceptable salt or prodrug thereof, wherein:
Cy is aryl, heteroaryl, cycloalkyl or heterocycloalkyl, each optionally substituted by 1, 2, 3, 4 or 5 —W—X—Y-Z;
R¹ is H, F, CN, OR⁵, SR⁵, C₁₋₆ alkyl, C₁₋₆ haloalkyl, C₂₋₁₂ alkoxyalkyl, C₂₋₁₂ haloalkoxyalkyl, cylcoalkyl, heterocycloalkyl, cycloalkylalkyl or heterocycloalkylalkyl;
R² is H, F, CN, C₁₋₆ alkyl, C₁₋₆ haloalkyl, C₂₋₁₂ alkoxyalkyl, C₂₋₁₂ haloalkoxyalkyl, cylcoalkyl, heterocycloalkyl, cycloalkylalkyl or heterocycloalkylalkyl;
wherein at least one of R¹ and R² is other than H;
R³ is H, C₁₋₆ alkyl, cycloalkyl or heterocycloalkyl, wherein each of the C₁₋₆ alkyl, cycloalkyl, and heterocycloalkyl is optionally substituted by 1, 2, 3, 4 or 5 —W′—X′—Y′-Z′;
R⁴ is C₁₋₆ alkyl, aryl, cycloalkyl, heteroaryl or heterocycloalkyl, each optionally substituted by 1, 2, 3, 4 or 5 —W′—X′—Y′-Z′;
or R³ and R⁴ together with the N atom to which they are attached form a 4-20 membered heterocycloalkyl group optionally substituted by 1, 2, 3, 4 or 5 —W′—X′—Y′-Z′;
each R⁵ is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl and heterocycloalkylalkyl is optionally substituted by 1, 2, 3, 4 or 5 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR³, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};
L is SO₂, (CR⁶R⁷)ₙ₁O(CR⁶R⁷)ₙ₂, (CR⁶R⁷)ₙ₁S(CR⁶R⁷)ₙ₂, or (CR⁶R⁷)ₙ₃
R⁶ and R⁷ are independently selected from H, halo, C₁₋₆ alkyl, C₁₋₆ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, and S(O)₂NR^{c′}R^{d′};
n1 is 0, 1, 2 or 3;
n2 is 0, 1, 2 or 3;
n3 is 1, 2, 3 or 4;
W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₈ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;
Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;
wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
each R^{a} and R^{a′} is independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;
each R^{b} and R^{b′} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group; and
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
provided that when R² is C₁₋₆ alkyl or C₁₋₆ haloalkyl, then R¹ is other than C₁₋₆ alkyl or C₁₋₆ haloalkyl.

2. The compound of claim 1, or pharmaceutically acceptable salt thereof, wherein Cy is aryl or heteroaryl, each optionally substituted by 1, 2, 3, 4 or 5 —W—X—Y-Z.

3. The compound of claim 1, or pharmaceutically acceptable salt thereof, wherein Cy is aryl or heteroaryl, each optionally substituted with 1, 2, 3, 4 or 5 —W—X—Y-Z wherein W is O or absent, X is absent, and Y is absent.

4. The compound of claim 1, or pharmaceutically acceptable salt thereof, wherein Cy is aryl optionally substituted by 1, 2, 3, 4 or 5 —W—X—Y-Z.

5. The compound of claim 1, or pharmaceutically acceptable salt thereof, wherein Cy is phenyl or naphthyl, each optionally substituted with 1, 2, 3, 4 or 5 —W—X—Y-Z.

6. The compound of claim 1, or pharmaceutically acceptable salt thereof, wherein Cy is phenyl or naphthyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₂₋₈ alkoxyalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, C₁₋₆ cyanoalkyl, C₁₋₆ hydroxyalkyl, C₂₋₈ alkoxyalkyl, CN, NO₂, OR^{a}, SR^{a}, C(O)NR^{c}R^{d}, NR^{c}C(O)R^{d} and COOR^{a}.

7. The compound of claim 1, or pharmaceutically acceptable salt thereof, wherein Cy is heteroaryl optionally substituted by 1, 2, 3, 4 or 5 —W—X—Y-Z.

8. The compound of claim 1, or pharmaceutically acceptable salt thereof, wherein Cy is pyridyl, pyrimidinyl, triazinyl, furanyl, thiazolyl, pyrazinyl, purinyl, quinazolinyl, quinolinyl, isoquinolinyl, pyrrolo[2,3-d]pyrimidinyl, or 1,3-benzothiazolyl, each optionally substituted by 1, 2, 3, 4 or 5 —W—X—Y-Z.

9. The compound of claim 1, or pharmaceutically acceptable salt thereof, wherein Cy is pyridyl, pyrimidinyl, triazinyl, furanyl, thiazolyl, pyrazinyl, purinyl, quinazolinyl, quinolinyl, isoquinolinyl, pyrrolo[2,3-d]pyrimidinyl, or 1,3-benzothiazolyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₂₋₈ alkoxyalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, C₁₋₆ cyanoalkyl, C₁₋₆ hydroxyalkyl, C₂₋₈ alkoxyalkyl, CN, NO₂, OR^{a}, SR^{a}, C(O)NR^{c}R^{d}, NR^{c}C(O)R^{d} and COOR^{a}.

10. The compound of claim 1, or pharmaceutically acceptable salt thereof, wherein Cy is cycloalkyl or heterocycloalkyl, each optionally substituted by 1, 2, 3, 4 or 5 —W—X—Y-Z.

11. The compound of claim 1, or pharmaceutically acceptable salt thereof, wherein Cy is cycloalkyl or heterocycloalkyl, each optionally substituted by 1, 2, 3, 4 or 5 —W—X—Y-Z wherein W is O or absent, X is absent, and Y is absent.

12. The compound of claim 1, or pharmaceutically acceptable salt thereof, wherein Cy is cyclopropyl, cyclobutyl, cyclopentyl, cyclohexyl, cyclocheptyl, adamantyl, aziridinyl, azetidinyl, pyrrolidine, piperidinyl, piperizinyl or morpholinyl, each optionally substituted by 1, 2, 3, 4 or 5 —W—X—Y-Z.

13. The compound of claim 1, or pharmaceutically acceptable salt thereof, wherein Cy is cyclopropyl, cyclobutyl, cyclopentyl, cyclohexyl, cyclocheptyl, adamantyl, aziridinyl, azetidinyl, pyrrolidine, piperidinyl, piperizinyl or morpholinyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₂₋₈ alkoxyalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, C₁₋₆ cyanoalkyl, C₁₋₆ hydroxyalkyl, C₂₋₈ alkoxyalkyl, CN, NO₂, OR^{a}, SR^{a}, C(O)NR^{c}R^{d}, NR^{c}C(O)R^{d} and COOR^{a}.

14. The compound of claim 1, or pharmaceutically acceptable salt thereof, wherein R¹ is H, OR⁵, SR⁵ or C₁₋₆ alkyl; and each R⁵is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl.

15. The compound of claim 1, or pharmaceutically acceptable salt thereof, wherein R¹ is H.

16. The compound of claim 1, or pharmaceutically acceptable salt thereof, wherein R¹ is OR⁵ or SR⁵.

17. The compound of claim 1, or pharmaceutically acceptable salt thereof, wherein R¹ is OR⁵.

18. The compound of claim 1, or pharmaceutically acceptable salt thereof, wherein R¹ is OR⁵ or SR⁵; and each R⁵ is independently H or C₁₋₆ alkyl.

19. The compound of claim 1, or pharmaceutically acceptable salt thereof, wherein R¹ is hydroxy, methoxy, or methylthio.

20. The compound of claim 1, or pharmaceutically acceptable salt thereof, wherein R² is H, C₁₋₆ alkyl or C₁₋₆ haloalkyl.

21. The compound of claim 1, or pharmaceutically acceptable salt thereof, wherein R² is methyl or ethyl.

22. The compound of claim 1, or pharmaceutically acceptable salt thereof, wherein R² is methyl.

23. The compound of claim 1, or pharmaceutically acceptable salt thereof, wherein R² is H.

24. The compound of claim 1, or pharmaceutically acceptable salt thereof, wherein R³ is H, C₁₋₆ alkyl, C₁₋₆ haloalkyl, C₁₋₆ hydroxyalkyl, C₂₋₁₂ alkoxyalkyl.

25. The compound of claim 1, or pharmaceutically acceptable salt thereof, wherein R³ is H or C₁₋₆ alkyl.

26. The compound of claim 1, or pharmaceutically acceptable salt thereof, wherein R³ is C₁₋₆ alkyl.

27. The compound of claim 1, or pharmaceutically acceptable salt thereof, wherein R⁴ is C₁₋₆ alkyl, cycloalkyl or heterocycloalkyl, each optionally substituted by 1, 2, 3, 4 or 5 —W′—X′—Y′-Z′.

28. The compound of claim 1, or pharmaceutically acceptable salt thereof, wherein R⁴ is cycloalkyl optionally substituted by 1, 2, 3, 4 or 5 —W′—X′—Y′-Z′.

29. The compound of claim 1, or pharmaceutically acceptable salt thereof, wherein R⁴ is heterocycloalkyl optionally substituted by 1, 2, 3, 4 or 5 —W′—X′—Y′-Z′.

30. The compound of claim 1 wherein:
R³ is H, C₁₋₆ alkyl, cycloalkyl or heterocycloalkyl, wherein each of the C₁₋₆ alkyl, cycloalkyl, and heterocycloalkyl is optionally substituted by 1, 2, 3, 4 or 5 —W′—X′—Y′-Z′; and
R⁴ is C₁₋₆ alkyl, aryl, cycloalkyl, heteroaryl or heterocycloalkyl, each optionally substituted by 1, 2, 3, 4 or 5 —W′—X′—Y′-Z′.

31. The compound of claim 1, or pharmaceutically acceptable salt thereof, wherein:
R³ is H or C₁₋₆ alkyl; and
R⁴ is cycloalkyl or heterocycloalkyl, each optionally substituted by 1, 2, 3, 4 or 5 —W′—X′—Y′-Z′.

32. The compound of claim 1, or pharmaceutically acceptable salt thereof, wherein R³ and R⁴ together with the N atom to which they are attached form a 5-14 membered heterocycloalkyl group optionally substituted by 1, 2, 3, or 4 —W′—X′—Y′-Z′.

33. The compound of claim 1, or pharmaceutically acceptable salt thereof, wherein R³ and R⁴ together with the N atom to which they are attached form a 4-7 membered heterocycloalkyl group optionally substituted by 1, 2, 3, or 4 —W′—X′—Y′-Z′.

34. The compound of claim 1, or pharmaceutically acceptable salt thereof, wherein R³ and R⁴ together with the N atom to which they are attached form a piperidinyl or pyrrolidinyl group optionally substituted by 1, 2, 3, or 4 —W′—X′—Y′-Z′.

35. The compound of claim 1, or pharmaceutically acceptable salt thereof, wherein R³ and R⁴ together with the N atom to which they are attached form a piperidinyl or pyrrolidinyl group substituted by 2, 3, or 4 —W′—X′—Y′-Z′; wherein two —W′—X′—Y′-Z′ are attached to the same atom and optionally form a 3-20 membered cycloalkyl or heterocycloalkyl group optionally substituted by 1, 2 or 3 —W″—X″—Y″-Z″.

36. The compound of claim 1, or pharmaceutically acceptable salt thereof, wherein each —W—X—Y-Z is independently selected from halo, cyano, C₁₋₆ cyanoalkyl, nitro, C₁₋₈ alkyl, C₂₋₈ alkenyl, C₁₋₈ haloalkyl, C₁₋₆ alkylthio, C₁₋₆ haloalkylthio, C₁₋₈ alkoxy, C₂₋₈ alkenyloxy, C₁₋₆ haloalkoxy, OH, (C₁₋₆ alkoxy)-C₁₋₆ alkyl, amino, C₁₋₆ alkylamino, C₂₋₈ dialkylamino, OC(O)NR^{c}R^{d}, NR^{c}C(O)R^{d}, NR^{c}C(O)OR^{a}, NR^{c}S(O)₂R^{d}, C(O)OR^{a}, C(O)R^{a}, C(O)NR^{a}NR^{c}R^{d}, S(O)₂R^{d}, SR^{d}, C(O)NR^{c}R^{d}, C(S)NR^{c}R^{d}, aryloxy, heteroaryloxy, cycloalkyloxy, cycloalkenyloxy, heterocycloalkyloxy, arylalkyloxy, heteroarylalkyloxy, cycloalkylalkyloxy, heterocycloalkylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl , heteroarylalkynyl, cycloalkylalkyl, and heterocycloalkylalkyl;
wherein each of the C₁₋₈ alkyl, C₂₋₈ alkenyl, C₁₋₈ haloalkyl, C₁₋₆ alkylthio, C₁₋₆ haloalkylthio, C₁₋₈ alkoxy, aryloxy, heteroaryloxy, arylalkyloxy, heteroarylalkyloxy, heteroaryloxyalkyl, aryloxy, heteroaryloxy, cycloalkyloxy, cycloalkenyloxy, heterocycloalkyloxy, arylalkyloxy, heteroarylalkyloxy, cycloalkylalkyloxy, heterocycloalkylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl, heteroarylalkynyl, cycloalkylalkyl, and heterocycloalkylalkyl is optionally substituted by 1, 2, or 3 halo, cyano, nitro, C₁₋₆ hydroxyalkyl, C₁₋₆ cyanoalkyl, aminoalkyl, dialkylaminoalkyl, C₁₋₆ alkyl, C₁₋₆ haloalkyl, C₁₋₆ cyanoalkyl, C₁₋₆ alkoxy, C₁₋₆ haloalkoxy, OH, OR^{a}, (C₁₋₆ alkoxy)-C₁₋₆ alkyl, amino, C₁₋₆ alkylamino, C₂₋₈ dialkylamino, C(O)NR^{c}R^{d}, C(O)OR^{a}, C(O)R^{a}, (cyclocalkylalkyl)-C(O)—, NR^{c}C(O)R^{d}, NR^{c}C(O)OR^{a}, NR^{c}S(O)₂R^{d}, C(S)NR^{c}R^{d}, S(O)₂R^{d}, SR^{d}, (C₁₋₆ alkyl)sulfonyl, arylsulfonyl, aryl optionally substituted by halo, heteroaryl, cycloalkylalkyl, cycloalkyl, or heterocycloalkyl.

37. The compound of claim 1, or pharmaceutically acceptable salt thereof, wherein each —W—X—Y-Z is independently selected from halo, cyano, C₁₋₆ cyanoalkyl, nitro, C₁₋₈ alkyl, C₁₋₈ alkenyl, C₁₋₈ haloalkyl, C₁₋₈ alkoxy, C₁₋₆ haloalkoxy, OH, C₁₋₈ alkoxyalkyl, amino, C₁₋₆ alkylamino, C₂₋₈ dialkylamino, OC(O)NR^{c}R^{d}, NR^{c}C(O)R^{d}, NR^{c}C(O)OR^{a}, aryloxy, heteroaryloxy, arylalkyloxy, heteroarylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl, heteroarylalkynyl, cycloalkylalkyl, or heterocycloalkylalkyl;
wherein each of the C₁₋₈ alkyl, C₁₋₈ alkenyl, C₁₋₈ haloalkyl, C₁₋₈ alkoxy, aryloxy, heteroaryloxy, arylalkyloxy, heteroarylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl, heteroarylalkynyl, cycloalkylalkyl, and heterocycloalkylalkyl is optionally substituted by 1, 2, or 3 substituents independently selected from halo, cyano, nitro, C₁₋₆ hydroxyalkyl, C₁₋₆ cyanoalkyl, aminoalkyl, dialkylaminoalkyl, C₁₋₆ alkyl, C₁₋₆haloalkyl, C₁₋₆ alkoxy, C₁₋₆ haloalkoxy, OH, C₁₋₈ alkoxyalkyl, amino, C₁₋₆ alkylamino, C₂₋₈ dialkylamino, C(O)NR^{c}R^{d}, C(O)OR^{a}, NR^{c}C(O)R^{d}, NR^{c}S(O)₂R^{d}, (C₁₋₆ alkyl)sulfonyl, arylsulfonyl, aryl, heteroaryl, cycloalkyl, and heterocycloalkyl.

38. The compound of claim 1, or pharmaceutically acceptable salt thereof, wherein each —W—X—Y-Z is independently selected from halo, C₁₋₆ alkyl, C₁₋₆ alkoxy, C₁₋₆ haloalkyl, C₁₋₆ haloalkoxy, aryl and heteroaryl, wherein each of the aryl and heteroaryl is optionally substituted by 1, 2, or 3 substituents independently selected from halo, cyano, nitro, C₁₋₆ hydroxyalkyl, C₁₋₆ cyanoalkyl, aminoalkyl, dialkylaminoalkyl, C₁₋₆ alkyl, C₁₋₆haloalkyl, C₁₋₆ alkoxy, C₁₋₆ haloalkoxy, OH, C₂₋₁₂ alkoxyalkoxy, C₂₋₁₂ alkoxyalkyl, amino, C₁₋₆ alkylamino, C₂₋₈ dialkylamino, C(O)NR^{c}R^{d}, C(O)OR^{a}, NR^{c}C(O)R^{d}, NR^{c}S(O)₂R^{d}, (C₁₋₆ alkyl)sulfonyl, arylsulfonyl, aryl, heteroaryl, cycloalkyl, and heterocycloalkyl.

39. The compound of claim 1, or pharmaceutically acceptable salt thereof, wherein each —W—X—Y-Z is independently selected from halo, cyano, C₁₋₆ cyanoalkyl, nitro, C₁₋₆ nitroalkyl, C₁₋₆ alkyl, C₁₋₆ haloalkyl, C₁₋₆ alkoxy, C₁₋₆ haloalkoxy, OH, (C₁₋₆ alkoxy)-C₁₋₆alkyl, amino, C₁₋₆ alkylamino, C₂₋₈ dialkylamino, aryl, heteroaryl, cycloalkyl, heterocycloalkyl, arylalkyl, heteroarylalkyl, cycloalkylalkyl, and heterocycloalkylalky.

40. The compound of claim 1, or pharmaceutically acceptable salt thereof, wherein:
each —W′—X′—Y′-Z′ is independently selected from halo, OH, cyano, CHO, COOH, C(O)O—(C₁₋₆ alkyl), C(O)—(C₁₋₆ alkyl), SO₂—(C₁₋₆ alkyl), C₁₋₆ alkyl, C₁₋₆ alkoxy and -L-R⁷, wherein the C₁₋₆ alkyl or C₁₋₆ alkoxy is optionally substituted by 1, 2, 3, 4, or 5 substituents independently selected from halo, OH, COOH and C(O)O—(C₁₋₆ alkyl);
L is absent, O, CH₂, NHSO₂, or N[C(O)—(C₁₋₆ alkyl)]; and
R⁷ is aryl or heteroaryl, each optionally substituted by 1, 2, or 3 substituents independently selected from halo, OH, cyano, CHO, COOH, C(O)O—(C₁₋₆ alkyl), C(O)—(C₁₋₆ alkyl), SO₂—(C₁₋₆ alkyl), SO₂—NH(C₁₋₆ alkyl), C₁₋₆ alkyl, C₁₋₆ alkoxy, C₁₋₆ haoalkyl, C₁₋₆ hydroxyalkyl, aryl, heteroaryl and aryloxy.

41. The compound of claim 1, or pharmaceutically acceptable salt thereof, wherein each —W′—X′—Y′-Z′ is indepently halo; C₁₋₆ alkyl; C₁₋₆ haloalkyl; OH; C₁₋₆ alkoxy; C₁₋₆ haloalkoxy; C₂₋₁₂ alkoxyalkoxy; C₁₋₆ hydroxyalkyl; C₂₋₁₂ alkoxyalkyl; aryl; heteroaryl; aryl substituted by halo, C₁₋₆ alkyl, C₁₋₆ alkoxy, aryl, heteroaryl, or aryloxy; or heteroaryl substituted by halo, C₁₋₆ alkyl, C₁₋₆ alkoxy, aryl, or heteroaryl.

42. The compound of claim 1, or pharmaceutically acceptable salt thereof, wherein two —W′—X′—Y′-Z′ are attached to the same atom and optionally form a 3-20 membered cycloalkyl or heterocycloalkyl group optionally substituted by 1, 2 or 3 —W″—X″—Y″-Z″.

43. The compound of claim 1, or pharmaceutically acceptable salt thereof, wherein each —W″—X″—Y″-Z″ is indepently halo, cyano, C₁₋₆ cyanoalkyl, nitro, C₁₋₆ nitroalkyl, C₁₋₆ alkyl, C₁₋₆ haloalkyl, C₁₋₆ alkoxy, C₁₋₆ haloalkoxy, OH, (C₁₋₆ alkoxy)-C₁₋₆alkyl, amino, C₁₋₆ alkylamino, C₂₋₈ dialkylamino, aryl, heteroaryl, cycloalkyl, heterocycloalkyl, arylalkyl, heteroarylalkyl, cycloalkylalkyl, or heterocycloalkylalkyl.

44. A compound of claim 1 having Formula II: 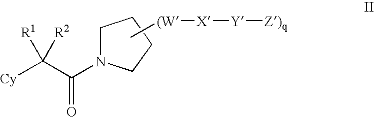
or pharmaceutically acceptable salt or prodrug thereof, wherein:
Cy is aryl, heteroaryl, cycloalkyl or heterocycloalkyl, each optionally substituted by 1, 2, 3, 4 or 5 —W—X—Y-Z;
R¹ is H, OR⁵ or SR⁵;
R² is H, C₁₋₆ alkyl or C₁₋₆ haloalkyl;
each R⁵ is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl and heterocycloalkylalkyl is optionally substituted by 1, 2, 3, 4 or 5 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};
W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₈ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;
Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{c}, SO, SO₂, SONR^{e}, and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;
wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
each R^{a} is independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;
each R^{b} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl; and
q is 0, 1, 2, 3 or 4.

45. A compound of claim 1 having Formula III: 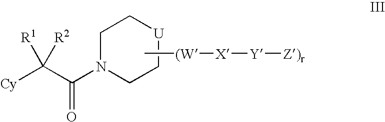
or pharmaceutically acceptable salt or prodrug thereof, wherein:
Cy is aryl, heteroaryl, cycloalkyl or heterocycloalkyl, each optionally substituted by 1, 2, 3, 4 or 5 —W—X—Y-Z;
U is NH, CH₂ or O;
R¹ is H, OR⁵ or SR⁵;
R² is H, C₁₋₆ alkyl or C₁₋₆ haloalkyl;
each R⁵ is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl and heterocycloalkylalkyl is optionally substituted by 1, 2, 3, 4 or 5 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};
W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₆ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;
Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{c}, and NR^{c}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;
wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
each R^{a} is independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;
each R^{b} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl; and
r is 0, 1, 2, 3 or 4.

46. A compound of claim 1 having Formula IV: 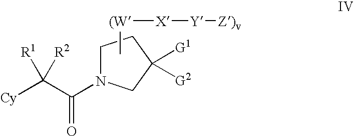
or pharmaceutically acceptable salt or prodrug thereof, wherein:
Cy is aryl, heteroaryl, cycloalkyl or heterocycloalkyl, each optionally substituted by 1, 2, 3, 4 or 5 —W—X—Y-Z;
R¹ is H, OR⁵ or SR⁵;
R² is H, C₁₋₆ alkyl or C₁₋₆ haloalkyl;
each R⁵ is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl and heterocycloalkylalkyl is optionally substituted by 1, 2, 3, 4 or 5 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};
G¹ and G² together with the carbon atom to which they are attached form a 3-20 membered cycloalkyl or heterocycloalkyl group optional substituted by 1, 2 or 3 —W″—X″—Y″-Z″.
W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₈ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;
Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;
wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
each R^{a} is independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;
each R^{b} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl; and
v is 0, 1 or 2.

47. A compound of claim 1 having Formula Va or Vb: 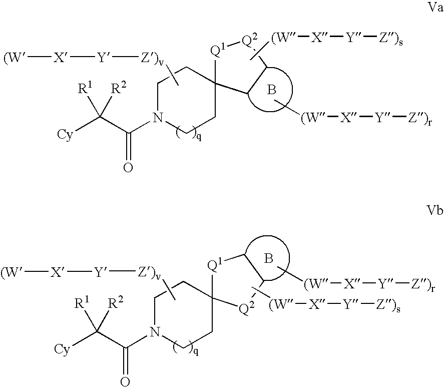
or pharmaceutically acceptable salt or prodrug thereof, wherein:
ring B is a fused 5 or 6-membered aryl or heteroaryl group;
Q¹ is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;
Q² is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;
Cy is aryl, heteroaryl, cycloalkyl or heterocycloalkyl, each optionally substituted by 1, 2, 3, 4 or 5 —W—X—Y-Z;
R¹ is H, OR⁵, SR⁵ or C₂₋₆ alkyl;
R² is H, C₁₋₆ alkyl or C₁₋₆ haloalkyl;
each R⁵ is independently H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl and heterocycloalkylalkyl is optionally substituted by 1, 2, 3, 4 or 5 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R , S(O)R^{b}, S(O)NR^{c}R^{d}, S(O)₂R^{b}, and S(O)₂NR^{c}R^{d};
W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₈ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;
Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkylk or 3-14 membered heterocycloalkyl group optionally substituted by 1, 2 or 3 —W″—X″—Y″-Z″;
wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
each R^{a} is independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;
each R^{b} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl; and
q is 0 or 1;
v is 0, 1 or 2;
r is 0, 1 or 2;
s is 0, 1 or 2; and
the sum of r and s is 0, 1 or 2.

48. A compound of claim 1 having Formula VI: 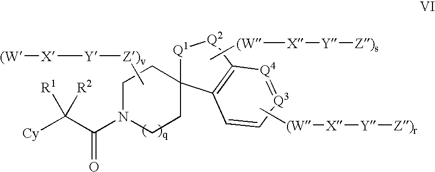
or pharmaceutically acceptable salt thereof, wherein:
Q¹ is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;
Q² is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;
Q³ and Q⁴ are independently selected from CH and N.
q is 0 or 1;
v is 0, 1 or 2;
r is 0, 1 or 2;
s is 0, 1 or 2; and
the sum of r and s is 0, 1 or 2.

49. A compound of claim 1 having Formula VII: 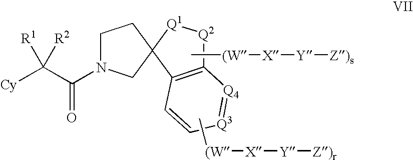
or pharmaceutically acceptable salt thereof, wherein:
Q¹ is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;
Q² is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;
Q³ and Q⁴ are independently selected from CH and N.
r is 0, 1 or 2;
s is 0, 1 or 2; and
the sum of r and s is 0, 1 or 2.

50. A compound of claim 1 having Formula VIII: 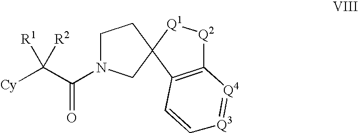
or pharmaceutically acceptable salt thereof, wherein:
Q¹ is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;
Q² is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH; and
Q³ and Q⁴ are independently selected from CH and N.

51. A compound of claim 1 having Formula IX: 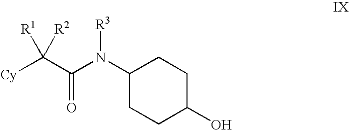
or pharmaceutically acceptable salt thereof.

52. A compound of claim 1, or pharmaceutically acceptable salt thereof, wherein the compound has Formula I.

53. A compound of claim 1 selected from:
1′-[(4-bromo-2-fluorophenyl)(hydroxy)acetyl]-3H-spiro[2-benzofuran-1,3′-pyrrolidin]-3-one;
1′-[(4-bromo-2-fluorophenyl)(methoxy)acetyl]-3H-spiro[2-benzofuran-1,3′-pyrrolidin]-3-one;
5-(3-fluoro-4-1-methoxy-2-oxo-2-[3-oxo-1′H,3H-spiro[2-benzofuran-1,3′-pyrrolidin]-1′-yl]ethylphenyl)-N-methylpyridine-2-carboxamide;
1′-[2-(4-bromo-2-fluorophenyl)-2-methoxybutanoyl]-3H-spiro[2-benzofuran-1,3′-pyrrolidin]-3-one;
2-(4-bromo-2-fluorophenyl)-2-hydroxy-N-methyl-N-(tetrahydro-2H-pyran-4-yl)acetamide;
1-(4-bromo-2-fluorophenyl)-2-oxo-2-(1,3,3-trimethyl-6-azabicyclo[3.2.1]oct-6-yl)ethanol;
6-[(4-bromo-2-fluorophenyl)(methoxy)acetyl]-1,3,3-trimethyl-6-azabicyclo[3.2.1]octane;
2-(4-bromophenyl)-N-(cis-4-hydroxycyclohexyl)-N-methylpropanamide;
8-[2-(4-bromophenyl)propanoyl]-8-azabicyclo[3.2.1]octan-3-ol;
1′-[2-(4-bromophenyl)propanoyl]-3H-spiro[2-benzofuran-1,3′-pyrrolidin]-3-one;
N-methyl-5-(4-1-methyl-2-oxo-2-[3-oxo-1H′,3H-spiro[2-benzofuran-1,3′-pyrrolidin]-1′-yl]ethylphenyl)pyridine-2-carboxamide;
5-(4-(2-[3-hydroxy-8-azabicyclo[3.2.1]oct-8-yl]-1-methyl-2-oxoethylphenyl))-N-methylpyridine-2-carboxamide;
5-(4-(2-[(4-hydroxycyclohexyl)(methyl)amino]-1-methyl-2-oxoethylphenyl))-N-methylpyridine-2-carboxamide;
2-(4-bromo-2-fluorophenyl)-2-fluoro-N-methyl-N-(tetrahydro-2H-pyran-4-yl)acetamide; and
6-[(4-bromo-2-fluorophenyl)(fluoro)acetyl]-1,3,3-trimethyl-6-azabicyclo[3.2.1]octane,
or a pharmaceutically acceptable salt thereof.

54. A compound of claim 1 selected from:
6-[(4-Bromo-2-fluorophenyl)(difluoro)acetyl]-1,3,3-trimethyl-6-azabicyclo[3.2.1]octane;
2-(4-Bromo-2-fluorophenyl)-1-oxo-1-(1,3,3-trimethyl-6-azabicyclo[3.2.1]oct-6-yl)propan-2-ol;
5-(3-fluoro-4-[1-fluoro-2-oxo-2-(1,3,3-trimethyl-6-azabicyclo[3.2.1]oct-6-yl)ethyl]phenyl)-N-methylpyridine-2-carboxamide;
5-(3-fluoro-4-[1-hydroxy-1-methyl-2-oxo-2-(1,3,3-trimethyl-6-azabicyclo[3.2.1]-oct-6-yl)ethyl]phenyl)-N-methylpyridine-2-carboxamide;
6-[2-(4-bromo-2-fluorophenyl)-2-methoxypropanoyl]-1,3,3-trimethyl-6-azabicyclo[3.2.1]octane; and
6-[2-(4-bromo-2-fluorophenyl)-2-fluoropropanoyl]-1,3,3-trimethyl-6-azabicyclo[3.2.1]octane; or pharmaceutically acceptable salt thereof.

55. A composition comprising a compound of claim 1, or pharmaceutically acceptable salt thereof, and at least one pharmaceutically acceptable carrier.

56. A method of modulating 11βHSD1 comprising contacting said 11βHSD1 with a compound of claim 1, or pharmaceutically acceptable salt thereof.

57. The method of claim 56 wherein said modulating is inhibiting.

58. A method of treating a disease in a patient, wherein said disease is associated with expression or activity of 11βHSD1, comprising administering to said patient a therapeutically effective amount of a compound of claim 1, or pharmaceutically acceptable salt thereof.

59. The method of claim 58 wherein said disease is obesity, diabetes, glucose intolerance, insulin resistance, hyperglycemia, atherosclerosis, hypertension, hyperlipidemia, cognitive impairment, dementia, depression, glaucoma, cardiovascular disorders, osteoporosis, inflammation, metabolic syndrome, coronary heart disease, type 2 diabetes, hypercortisolemia, androgen excess, or polycystic ovary syndrome (PCOS).

60. A method of treating metabolic syndrome in a patient comprising administering to said patient a therapeutically effective amount of a compound of claim 1, or pharmaceutically acceptable salt thereof.

61. A method of treating type 2 diabetes in a patient comprising administering to said patient a therapeutically effective amount of a compound of claim 1, or pharmaceutically acceptable salt thereof.
